(19) United States
(12) Patent Application Publication
(10) Pub. No.: US 2026/0159609 A1
(43) Pub. Date: Jun. 11, 2026

(54) IgG4 BINDING POLYPEPTIDES WITH IMPROVED FC FUNCTION

(71) Applicant: ABLYNX N.V., Zwijnaarde (BE)

(72) Inventors: Julie A. JAWORSKI, Bolton, MA (US); Sagar V. KATHURIA, Harvard, MA (US); Sunghae PARK, Waban, MA (US); Megan SALEMI, Cambridge, MA (US); Victor Zhao SUN, Cambridge, MA (US)

(21) Appl. No.: 19/383,125

(22) Filed: Nov. 7, 2025

Related U.S. Application Data
(60) Provisional application No. 63/718,210, filed on Nov. 8, 2024.

(51) Int. Cl. C07K 16/42

(52) U.S. Cl. CPC C07K 16/42; C07K 2317/71; C07K 2317/92

(57) ABSTRACT

Provided herein are variant IgG4 Fc domains comprising amino acid substitutions that decrease self-interaction of a polypeptide and/or decrease aggregation.

### CROSS REFERENCE TO RELATED APPLICATION

This application claims the benefit of U.S. Provisional Patent Application Ser. No. 63/718,210, filed Nov. 8, 2024. The entire content of the above-referenced patent application is incorporated by reference in its entirety herein.

### REFERENCE TO SEQUENCE LISTING SUBMITTED ELECTRONICALLY

The content of the electronically submitted sequence listing in XML file (771028_SA9-913_ST26.xml; Size: 74,363 bytes; Created: Nov. 7, 2025) is incorporated herein by reference in its entirety.

### BACKGROUND

Immunoglobulins and immunoglobulin derived molecules are proteins that play a pivotal role in a number of different disease therapies. Immunoglobulins possess a number of desirable characteristics that contribute to their usefulness as therapeutics. In addition to their use in FDA-approved monoclonal antibody therapies, Fc fusion proteins are also approved for use in a number of diseases.

Five classes of immunoglobulins are known to be expressed in mammals: IgA, IgD, IgE, IgG, and IgM. Of these, IgG molecules have received the most focus for their therapeutic use. Four different IgG subclasses are expressed in humans: IgG1, IgG2, IgG3, and IgG4. Each subclass has varying characteristics, specifically with respect to their effector functions. While IgG1 molecules make up the vast majority of monoclonal antibody therapeutics, IgG4 therapies have also been developed. IgG4 has a number of characteristics that are beneficial in certain contexts. For instance, they have reduced effector functions and they undergo Fab-arm exchange. IgG4 molecules also have lower antibody-dependent cell-mediated cytotoxicity (ADCC). These characteristics have made IgG4 an attractive candidate for therapies in which effector functions are not desirable. Despite ongoing efforts, there remains a need to engineer new IgG4-based molecules that exhibit characteristics more favorable for development into therapeutics.

### BRIEF SUMMARY

Certain aspects of the disclosure are directed to an isolated polypeptide comprising an Fc region from human IgG4, wherein the Fc region comprises substitutions at amino acid residues corresponding to amino acid residues Q274, Q355, and E419 according to EU numbering in human IgG4.

In some aspects, the glutamine at amino acid residue 274 according to EU numbering in human IgG4 is substituted by lysine (Q274K). In some aspects, the glutamine at amino acid residue 355 according to EU numbering in human IgG4 is substituted with arginine (Q355R). In some aspects, the glutamate at amino acid residue 419 according to EU numbering in human IgG4 is substituted by glutamine (E419Q). In some aspects, the glutamine at amino acid residue 274 according to EU numbering in human IgG4 is substituted by lysine (Q274K); the glutamine at amino acid residue 355 according to EU numbering in human IgG4 is substituted with arginine (Q355R); and the glutamate at amino acid residue 419 according to EU numbering in human IgG4 is substituted by glutamine (E419Q).

Other aspects of the disclosure are directed to an isolated polypeptide comprising an Fc region from human IgG4, wherein the Fc region further comprises a substitution at an amino acid residue corresponding to E294 according to EU numbering in human IgG4. In some aspects, the glutamate at amino acid residue 294 according to EU numbering in human IgG4 is substituted by glutamine (E294Q). In some aspects, the glutamine at amino acid residue 274 according to EU numbering in human IgG4 is substituted by lysine (Q274K); the glutamate at amino acid residue 294 according to EU numbering in human IgG4 is substituted by glutamine (E294Q); the glutamine at amino acid residue 355 according to EU numbering in human IgG4 is substituted with arginine (Q355R); and the glutamate at amino acid residue 419 according to EU numbering in human IgG4 is substituted by glutamine (E419Q).

Yet other aspects of the disclosure are directed to an isolated polypeptide comprising an Fc region from human IgG4, wherein the Fc region further comprises a substitution at the amino acid residue corresponding to R409 in human IgG4. In some aspects, the arginine at amino acid residue 409 according to EU numbering in human IgG4 according to EU numbering is substituted by lysine (R409K).

Other aspects of the disclosure are directed to an isolated polypeptide comprising an Fc region from human IgG4, wherein the Fc region further comprises a substitution at an amino acid residue corresponding to S228, L235, or both S228 and L235 in human IgG4 according to EU numbering. In certain aspects, the serine at amino acid residue 228 according to EU numbering in human IgG4 is substituted by proline (S228P), the leucine at amino acid residue 235 according to EU numbering in human IgG4 is substituted by glutamate (L235E), or the serine at amino acid residue 228 according to EU numbering in human IgG4 is substituted by proline (S228P) and the leucine at amino acid residue 235 according to EU numbering in human IgG4 is substituted by glutamate (L235E).

Other aspects of the disclosure are directed to an isolated polypeptide comprising an Fc region from human IgG4, wherein the Fc region further comprises a substitution at an amino acid residue corresponding to F234, L235, or both F234 and L235 in human IgG4. In some aspects, the phenylalanine at amino acid residue 234 according to EU numbering in human IgG4 is substituted by alanine (F234A), the leucine at amino acid residue 235 according to EU numbering in human IgG4 is substituted by alanine (L235A), or the phenylalanine at amino acid residue 234 according to EU numbering in human IgG4 is substituted by alanine (F234A) and the leucine at amino acid residue 235 according to EU numbering in human IgG4 is substituted by alanine (L235A). In some aspects, the Fc region further comprises a substitution at an amino acid residue corresponding to S228 in human IgG4 according to EU numbering. In some aspects, the serine at amino acid residue 228 according to EU numbering in human IgG4 is substituted by proline (S228P).

Other aspects of the disclosure are directed to an isolated polypeptide comprising an Fc region from human IgG4, the Fc region further comprises a substitution of the amino acid at an amino acid residue corresponding to S298 in human IgG4 according to EU numbering, a substitution of the amino acid at an amino acid residue corresponding to T299 in human IgG4 according to EU numbering, a substitution of the amino acid at an amino acid residue corresponding to Y300 in human IgG4 according to EU numbering, or a substitution at S298, T299, and Y300 in human IgG4 according to EU numbering. In some aspects, the serine at an amino acid residue corresponding to residue 298 in human IgG4 according to EU numbering is substituted with asparagine, wherein the threonine at an amino acid residue corresponding to residue 299 in human IgG4 according to EU numbering is substituted with alanine, and/or the tyrosine at an amino acid residue corresponding to residue 300 in human IgG4 according to EU numbering is substituted with serine.

Other aspects of the disclosure are directed to an isolated polypeptide comprising an Fc region from human IgG4, wherein the Fc region further comprises a substitution of the amino acid at an amino acid residue corresponding to T256 in human IgG4 according to EU numbering, a substitution of the amino acid at an amino acid residue corresponding to T307 in human IgG4 according to EU numbering, or a substitution at both T256 and T307 in human IgG4 according to EU numbering. In certain aspects, the threonine at an amino acid residue corresponding to residue 256 in human IgG4 according to EU numbering is substituted with aspartic acid, the threonine at an amino acid residue corresponding to residue 307 in human IgG4 according to EU numbering is substituted with glutamine, or wherein the threonine at an amino acid residue corresponding to residue 256 in human IgG4 according to EU numbering is substituted with aspartic acid and the threonine at an amino acid residue corresponding to residue 307 in human IgG4 according to EU numbering is substituted with glutamine.

Other aspects of the disclosure are directed to an isolated polypeptide comprising an Fc region from human IgG4, wherein the Fc region further comprises a substitution of the amino acid at an amino acid residue corresponding to M428 in human IgG4 according to EU numbering, a substitution of the amino acid at an amino acid residue corresponding to N434 in human IgG4 according to EU numbering, or a substitution at both M428 and N434 in human IgG4 according to EU numbering. In certain aspects, the methionine at an amino acid residue corresponding to residue 428 in human IgG4 according to EU numbering is substituted with leucine, wherein the asparagine at an amino acid residue corresponding to residue 434 in human IgG4 according to EU numbering is substituted with serine, or wherein the methionine at an amino acid residue corresponding to residue 428 in human IgG4 according to EU numbering is substituted with leucine and the asparagine at an amino acid residue corresponding to residue 434 in human IgG4 according to EU numbering is substituted with serine.

Certain aspects of the disclosure are directed to an isolated polypeptide comprising an Fc region from human IgG4, wherein the isolated polypeptide has decreased self-interaction as measured by dynamic light scattering compared to an isolated polypeptide comprising the Fc region without said substitutions. In some aspects, the isolated polypeptide has a diffusion interaction parameter equal to or greater than the diffusion interaction parameter of an Fc region from a wild-type IgG1 polypeptide. In some aspects, the isolated polypeptide has a diffusion interaction parameter equal to or greater than 20.0 mL/g when measured by dynamic light scattering. In some aspects, the isolated polypeptide has a diffusion interaction parameter equal to or greater than 30.0 mL/g when measured by dynamic light scattering. In some aspects, the isolated polypeptide has a diffusion interaction parameter of 31.8 mL/g or greater when measured by dynamic light scattering. In some aspects, the isolated polypeptide has a diffusion interaction parameter of 32.0 mL/g or greater when measured by dynamic light scattering. In some aspects, the isolated polypeptide has a diffusion interaction parameter equal to or greater than 41 mL/g when measured by dynamic light scattering. In some aspects, the isolated polypeptide has a diffusion interaction parameter of 41.5 mL/g or greater when measured by dynamic light scattering. In some aspects, the isolated polypeptide has a diffusion interaction parameter of 41.8 mL/g or greater when measured by dynamic light scattering.

Certain aspects of the disclosure are directed to an isolated polypeptide comprising an Fc region from human IgG4, wherein binding affinity to human neonatal Fc receptor (FcRn) is substantially identical to the binding affinity of an isolated polypeptide comprising a wildtype Fc region from human IgG4 to human FcRn. In some aspects, binding affinity to human Fc gamma receptor (FcγR) is substantially identical to the binding affinity of an isolated polypeptide comprising a wildtype Fc region from human IgG4 to human IgG4.

Certain aspects of the disclosure are directed to an isolated polypeptide comprising an Fc region from human IgG4, wherein the polypeptide is an antibody. In some aspects, the polypeptide is a monospecific antibody.

Certain aspects of the disclosure are directed to a composition or solution comprising an isolated polypeptide comprising an Fc region from human IgG4, wherein the composition or solution is less viscous than a composition or solution comprising an isolated polypeptide comprising a wildtype Fc region from human IgG4. In some aspects, the composition or solution has a viscosity of less than 20 centipoise (cP). In some aspects, the composition or solution has a viscosity of less than 15 centipoise (cP). In some aspects, the composition or solution has a viscosity of 13.9 centipoise (cP) or lower. In some aspects, the composition or solution has a viscosity of 12.2 centipoise (cP) or lower. In some aspects, the composition or solution has a viscosity of 8.5 centipoise (cP) or lower. In some aspects, the composition or solution has a viscosity of 7.9 centipoise (cP) or lower.

Certain aspects of the disclosure are directed to a composition or solution comprising an isolated polypeptide comprising an Fc region from human IgG4, wherein the composition or solution has an opalescence of less than or equal to 12 NTU. In some aspects, the composition or solution has an opalescence of 8.7 NTU or lower. In some aspects, the composition or solution has an opalescence of 7.3 NTU or lower. In some aspects, the composition or solution has an opalescence of 6.7 NTU or lower.

Certain aspects of the disclosure are directed to an isolated polypeptide comprising an amino acid sequence selected from the group consisting of SEQ ID NOs: 5-9, 12-17, 21, 22, 25-28, 31-34, 36-39, 41-44, and 46-66.

Certain aspects of the disclosure are directed to an isolated polypeptide comprising an Fc region from human IgG4, wherein the glutamine at amino acid residue 274 according to EU numbering in human IgG4 is substituted by lysine (Q274K); wherein the glutamine at amino acid residue 355 according to EU numbering in human IgG4 is substituted with arginine (Q355R); wherein the glutamate at amino acid residue 419 according to EU numbering in human IgG4 is substituted by glutamine (E419Q); wherein the serine at amino acid residue 228 according to EU numbering in human IgG4 is substituted by proline (S228P); wherein the threonine at an amino acid residue corresponding to residue 256 in human IgG4 according to EU numbering is substituted with aspartic acid (T256D); and wherein the threonine at an amino acid residue corresponding to residue 307 in human IgG4 according to EU numbering is substituted with glutamine (T307Q). In some embodiments, the arginine at amino acid residue 409 according to EU numbering in human IgG4 is substituted by lysine (R409K). In some embodiments, the leucine at amino acid residue 235 according to EU numbering in human IgG4 is substituted by glutamate (L235E). In some embodiments, the arginine at amino acid residue 409 according to EU numbering in human IgG4 is substituted by lysine (R409K). In some embodiments, the glutamate at amino acid residue 294 according to EU numbering in human IgG4 is substituted by glutamine (E294Q). In some embodiments, the glutamate at amino acid residue 294 according to EU numbering in human IgG4 is substituted by glutamine (E294Q) and the arginine at amino acid residue 409 according to EU numbering in human IgG4 is substituted by lysine (R409K). In some embodiments, the glutamate at amino acid residue 294 according to EU numbering in human IgG4 is substituted by glutamine (E294Q) and the leucine at amino acid residue 235 according to EU numbering in human IgG4 is substituted by glutamate (L235E). In some embodiments, the glutamate at amino acid residue 294 according to EU numbering in human IgG4 is substituted by glutamine (E294Q); the leucine at amino acid residue 235 according to EU numbering in human IgG4 is substituted by glutamate (L235E); and. the arginine at amino acid residue 409 according to EU numbering in human IgG4 is substituted by lysine (R409K).

Certain aspects of the disclosure are directed to an isolated polypeptide comprising an Fc region from human IgG4, wherein the glutamine at amino acid residue 274 according to EU numbering in human IgG4 is substituted by lysine (Q274K); wherein the glutamine at amino acid residue 355 according to EU numbering in human IgG4 is substituted with arginine (Q355R); wherein the glutamate at amino acid residue 419 ac-cording to EU numbering in human IgG4 is substituted by glutamine (E419Q); wherein the serine at amino acid residue 228 according to EU numbering in human IgG4 is substituted by proline (S228P); wherein the methionine at an amino acid residue corresponding to residue 428 in hu-man IgG4 according to EU numbering is substituted with leucine (M428L); and wherein the as-paragine at an amino acid residue corresponding to residue 434 in human IgG4 according to EU numbering is substituted with serine (N434S). In some embodiments, the arginine at amino acid residue 409 according to EU numbering in human IgG4 is substituted by lysine (R409K). In some embodiments, the leucine at amino acid residue 235 according to EU numbering in human IgG4 is substituted by glutamate (L235E). In some embodiments, the leucine at amino acid residue 235 according to EU numbering in human IgG4 is substituted by glutamate (L235E) and the arginine at amino acid residue 409 according to EU numbering in human IgG4 is substituted by lysine (R409K). In some embodiments, the glutamate at amino acid residue 294 according to EU numbering in human IgG4 is substituted by glutamine (E294Q). In some embodiments, the glutamate at amino acid residue 294 according to EU numbering in human IgG4 is substituted by glutamine (E294Q) and the arginine at amino acid residue 409 according to EU numbering in human IgG4 is substituted by lysine (R409K). In some embodiments, the glutamate at amino acid residue 294 according to EU numbering in human IgG4 is substituted by glutamine (E294Q) and the leucine at amino acid residue 235 according to EU numbering in human IgG4 is substituted by glutamate (L235E). In some embodiments, the glutamate at amino acid residue 294 according to EU numbering in human IgG4 is substituted by glutamine (E294Q); the leucine at amino acid residue 235 according to EU numbering in human IgG4 is substituted by glutamate (L235E); and the arginine at amino acid residue 409 according to EU numbering in human IgG4 is substituted by lysine (R409K).

Certain aspects of the disclosure are directed to an isolated polypeptide comprising an Fc region from human IgG4, wherein the glutamine at amino acid residue 274 according to EU numbering in human IgG4 is substituted by lysine (Q274K); wherein the glutamine at amino acid residue 355 according to EU numbering in human IgG4 is substituted with arginine (Q355R); wherein the glutamate at amino acid residue 419 according to EU numbering in human IgG4 is substituted by glutamine (E419Q); wherein the serine at amino acid residue 228 according to EU numbering in human IgG4 is substituted by proline (S228P); and wherein the phenylalanine at amino acid residue 234 according to EU numbering in human IgG4 is substituted by alanine (F234A) and the leucine at amino acid residue 235 according to EU numbering in human IgG4 is substituted by alanine (L235A). In some embodiments, the methionine at an amino acid residue corresponding to residue 428 in human IgG4 according to EU numbering is substituted with leucine (M428L); and wherein the asparagine at an amino acid residue corresponding to residue 434 in human IgG4 according to EU numbering is substituted with serine (N434S). In some embodiments, the methionine at an amino acid residue corresponding to residue 428 in human IgG4 according to EU numbering is substituted with leucine (M428L); the asparagine at an amino acid residue corresponding to residue 434 in human IgG4 according to EU numbering is substituted with serine (N434S); and the arginine at amino acid residue 409 according to EU numbering in human IgG4 is substituted by lysine (R409K). In some embodiments, the methionine at an amino acid residue corresponding to residue 428 in human IgG4 according to EU numbering is substituted with leucine (M428L); the asparagine at an amino acid residue corresponding to residue 434 in human IgG4 according to EU numbering is substituted with serine (N434S); and the glutamate at amino acid residue 294 according to EU numbering in human IgG4 is substituted by glutamine (E294Q). In some embodiments, the methionine at an amino acid residue corresponding to residue 428 in human IgG4 according to EU numbering is substituted with leucine (M428L); the asparagine at an amino acid residue corresponding to residue 434 in human IgG4 according to EU numbering is substituted with serine (N434S); the glutamate at amino acid residue 294 according to EU numbering in human IgG4 is substituted by glutamine (E294Q); and the arginine at amino acid residue 409 according to EU numbering in human IgG4 is substituted by lysine (R409K). In some embodiments, the threonine at an amino acid residue corresponding to residue 256 in human IgG4 according to EU numbering is substituted with aspartic acid (T256D); and wherein the threonine at an amino acid residue corresponding to residue 307 in human IgG4 according to EU numbering is substituted with glutamine (T307Q). In some embodiments, the threonine at an amino acid residue corresponding to residue 256 in human IgG4 according to EU numbering is substituted with aspartic acid (T256D); the threonine at an amino acid residue corresponding to residue 307 in human IgG4 according to EU numbering is substituted with glutamine (T307Q); and wherein the arginine at amino acid residue 409 according to EU numbering in human IgG4 is substituted by lysine (R409K). In some embodiments, the threonine at an amino acid residue corresponding to residue 256 in human IgG4 according to EU numbering is substituted with aspartic acid (T256D); the threonine at an amino acid residue corresponding to residue 307 in human IgG4 according to EU numbering is substituted with glutamine (T307Q); the glutamate at amino acid residue 294 according to EU numbering in human IgG4 is substituted by glutamine (E294Q). In some embodiments, the threonine at an amino acid residue corresponding to residue 256 in human IgG4 according to EU numbering is substituted with aspartic acid (T256D); the threonine at an amino acid residue corresponding to residue 307 in human IgG4 according to EU number-ing is substituted with glutamine (T307Q); the glutamate at amino acid residue 294 according to EU numbering in human IgG4 is substituted by glutamine (E294Q); and wherein the arginine at amino acid residue 409 according to EU numbering in human IgG4 is substituted by lysine (R409K).

Also disclosed herein are pharmaceutical compositions comprising any of the isolated polypeptides disclosed herein and a pharmaceutically acceptable carrier.

Also disclosed herein are methods of treating a disease or disorder, the method comprising ad-ministering an effective amount of any of the isolated polypeptides of the disclosure to a subject in need thereof.

Also disclosed herein are polynucleotides or sets of polynucleotides encoding any of the isolated polypeptides disclosed herein.

Also disclosed herein are vectors comprising the polynucleotides or sets of polynucleotides encoding any of the isolated polypeptides disclosed herein, and one or more promoter operably linked to the polynucleotides or sets of polynucleotides.

Also disclosed are host cells comprising the polynucleotides disclosed herein. In some embodiments, the host cell is a mammalian cell.

Also disclosed herein are methods of making any of the isolated polypeptides disclosed herein, the methods comprising transfecting one or more host cell with a polynucleotide or vector encoding the polypeptide and expressing the polypeptide in the host cell.

### BRIEF DESCRIPTION OF THE DRAWINGS

FIG. 1 is a visual representation of the structure of IgG4 showing surface exposed residues Q274, Q355, and E419, as well as the residue R409, according to EU numbering in a human IgG4 Fc.

FIG. 2 is a sequence alignment comparison showing the relative corresponding sequences between an IgG1 full constant region sequence according to SEQ ID NO: 1, an IgG4_PE full constant region sequence of SEQ ID NO: 9, and an IgG4 sequence with the R409K and the “Quad” (Q274K/E294Q/Q355R/E419Q) substitutions (SEQ ID NO: 13).

FIG. 3 A-L are a series of sensorgrams displaying the results of Surface Plasmon Resonance (SPR) assays to measure binding of IgG4 Fc regions with amino acid substitutions to the human neonatal Fc Receptor (FcRn) or the mouse FcRn. Binding of human FcRn with the Fc region of human IgG4 (SEQ ID NO: 4) comprising substitutions S228P/L235E (IgG4_PE; FIG. 3A), S228P/L235E/R409K (IgG4_PE_R409K; FIG. 3C), S228P/L235E/Q274K/Q355R/E419Q (IgG4_PE_Triple; FIG. 3E), S228P/L235E/Q274K/Q355R/E419Q/R409K (IgG4_PE_Triple_R409K; FIG. 3G), S228P/L235E/Q274K/E294Q/Q355R/E419Q (IgG4_PE_Quad; FIG. 3I), and S228P/L235E/Q274K/E294Q/Q355R/E419Q/R409K (IgG4_PE_Quad_R409K; FIG. 3K) were analyzed by SPR and measured in relative response units (RU). Binding of mouse FcRn with human IgG4 Fc region (SEQ ID NO: 4) comprising substitutions at S228P/L235E (IgG4_PE; FIG. 3B), S228P/L235E/R409K (IgG4_PE_R409K; FIG. 3D), S228P/L235E/Q274K/Q355R/E419Q (IgG4_PE_Triple; FIG. 3F), S228P/L235E/Q274K/Q355R/E419Q/R409K (IgG4_PE_Triple_R409K; FIG. 3H), S228P/L235E/Q274K/E294Q/Q355R/E419Q (IgG4_PE_Quad; FIG. 3J), and S228P/L235E/Q274K/E294Q/Q355R/E419Q/R409K (IgG4_PE_Quad_R409K; FIG. 3L) were analyzed by SPR and measured as a plot of relative response vs time. A 1:2 6-point dilution series of human or mouse FcRn starting at 1.5 μM or 1 μM, respectively, was injected for 50 sec over captured antibody, followed by 60 sec dissociation in PBST pH 5.8 running buffer.

FIG. 4 A-D are graphical representations of the results of a mock viral inactivation assay at various pH levels. FIG. 4A displays the percent aggregation of BR-D when appended to various Ig Fc regions. FIG. 4B displays the percent aggregation of the binding region of BR-E when appended to various Ig Fc regions.

FIG. 4C displays the percent aggregation of the binding region of BR-B when appended to various Ig Fc regions. FIG. 4D displays the percent aggregation of the binding region of BR-C when appended to various Ig Fc regions.

### DETAILED DESCRIPTION

The present disclosure provides novel Fc domain variants of immunoglobulin G4 (IgG4) having improved characteristics. The present disclosure further provides novel Fc domain variants of IgG4 comprising substitutions at amino acid residues corresponding to amino acid residues Q274, Q355, and E419 according to EU numbering in human IgG4. The present disclosure further provides novel Fc domain variants of IgG4 comprising substitutions at amino acid residues corresponding to amino acid residues Q274, E294, Q355, and E419 according to EU numbering in human IgG4. The present disclosure further provides novel Fc domain variants of IgG4 comprising substitutions that reduce self-interaction.

The present disclosure also provides nucleic acids encoding Fc domain variants (e.g., novel binding polypeptides comprising Fc domain variants), recombinant expression vectors and host cells for making Fc domain variants (e.g., novel binding polypeptides comprising Fc domain variants), methods of making the polypeptide (e.g., transfecting a host cell with a nucleic acid encoding the polypeptide), and pharmaceutical compositions comprising the isolated Fc domain variants (e.g., novel binding polypeptides comprising Fc domain variants). Methods of using the Fc domain variants (e.g., novel binding polypeptides comprising Fc domain variants) of the present disclosure to treat one or more diseases or disorders are also provided.

### Definitions

Unless stated otherwise, terms and techniques used within this application have the meaning generally known to one of skill in the art. It is to be understood that the terminology used herein is for the purpose of describing particular embodiments only and is not intended to be limiting.

Unless otherwise required by context, singular terms shall include pluralities and plural terms shall include the singular. The use of “or” means “and/or” unless stated otherwise. The use of the term “including,” as well as other forms, such as “includes” and “included,” is not limiting. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. Any single term, single element, single phrase, group of terms, group of phrases, or group of elements described herein can each be specifically excluded from the claims.

Generally, nomenclature used in connection with cell culture, molecular biology, immunology, microbiology, genetics, protein biology, and chemistry described herein is well-known and commonly used in the art. The nomenclatures used in connection with, and the laboratory procedures and techniques of, analytical chemistry, synthetic organic chemistry, and medicinal and pharmaceutical chemistry described herein are those well-known and commonly used in the art. For the disclosure to be more readily understood, select terms are defined below.

The term “polypeptide” refers to any polymeric chain of amino acids and encompasses native or artificial proteins, polypeptide analogs or variants of a protein sequence, or fragments thereof, unless otherwise contradicted by context. A polypeptide can be monomeric or polymeric. For a polypeptide (e.g., a Fc domain variant polypeptide comprising at least one substitution, according to EU numbering, as compared to a Fc domain parent polypeptide) a fragment of a polypeptide optionally contains at least one contiguous or nonlinear epitope of a polypeptide. A fragment polypeptide can be about 25, 50, 75, 100, 150, 200, 250, 300, 350, 400 or more amino acids in length. The precise boundaries of the at least one epitope fragment can be confirmed using ordinary skill in the art. A polypeptide fragment comprises at least about 5 contiguous amino acids, at least about 10 contiguous amino acids, at least about 15 contiguous amino acids, or at least about 20 contiguous amino acids, at least about 50 contiguous amino acids, at least about 100 contiguous amino acids, at least about 150 contiguous amino acids, at least about 200 contiguous amino acids, at least about 250 contiguous amino acids, at least about 300 contiguous amino acids, at least about 400 contiguous amino acids for example. A fragment polypeptide may also be a domain or part of a domain of a parent polypeptide.

The term “isolated polypeptide” refers to a protein or polypeptide that by virtue of its origin or source of derivation is not associated with naturally associated components that accompany it in its native state; is substantially free of other proteins from the same species. No particular level of purification is required. For example, an isolated polypeptide can simply be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique. An isolated recombinant polypeptide can be expressed by a cell that does not naturally express the recombinant polypeptide. In some aspects, an isolated polypeptide does not occur in nature. A protein or polypeptide that is chemically synthesized or synthesized in a cellular system can be different from the cell from which it naturally originates and therefore will be “isolated” from its naturally associated components. A protein or polypeptide can also be rendered substantially free of naturally associated components by isolation using protein purification techniques.

In certain aspects of the disclosure, Fc domains, e.g., Fc domain variants, are provided. As used herein, the term “Fc region” or “Fc domain” refers to the portion of a heavy chain constant region beginning in the hinge region and ending at the C-terminus of the antibody. Accordingly, a complete Fc region comprises at least a partial hinge domain, a CH2 domain, and a CH3 domain. The disclosure provides for complete Fc regions and partial Fc regions.

As used herein, the term “native residue,” “wild-type residue,” “parental residue” refers to an amino acid residue that occurs naturally at a particular amino acid position of a binding polypeptide (e.g., a wild-type IgG Fc domain) and which has not been modified, introduced, or altered by the hand of man. Accordingly, the “parental polypeptide” can refer to a wild-type amino acid sequence encoding a binding polypeptide (e.g., a wild-type IgG Fc domain). The “parental polypeptide” can refer to an amino acid sequence which has been modified but still serves as the reference binding polypeptide when compared to the variant binding polypeptide. For example, a Fc domain parent polypeptide can be a humanized IgG Fc domain.

As used herein, the term “substitution,” in the context of an amino acid or an amino acid sequence, refers to a difference in one or more amino acids between a sequence of interest and a reference sequence. The terms “substitution” and “mutation” may be used interchangeably. Except where otherwise noted, substitution does not refer to a deletion, in which an amino acid is absent from a sequence of interest when compared to a reference sequence (e.g., the amino acid sequence SSSS has a one amino acid deletion when compared to the reference sequence SSSSS).

As used herein, the term “self-interaction” refers to interaction of two polypeptides As used herein, the term “self-interaction substitution” refers to a substitution of one amino acid in a reference amino acid sequence for a different amino acid, such that two polypeptides comprising the resulting substitution sequence are less likely to interact with each other than two polypeptides comprising the reference sequence.

As known in the art, “sequence identity” between two polypeptides is determined by comparing the amino acid sequence of one polypeptide to the sequence of a second polypeptide. When discussed herein, whether any particular polypeptide is at least about 50%, 60%, 70%, 75%, 80%, 85%, 90%, 95%, 99%, or 100% identical to another polypeptide can be determined using methods and computer programs/software known in the art such as, but not limited to, the BESTFIT program (Wisconsin Sequence Analysis Package, Version 8 for Unix, Genetics Computer Group, University Research Park, 575 Science Drive, Madison, WI 5371 1). BESTFIT uses the local homology algorithm of Smith and Waterman, Advances in Applied Mathematics 2:482-489 (1981), to find the best segment of homology between two sequences. When using BESTFIT or any other sequence alignment program to determine whether a particular sequence is, for example, 95% identical to a reference sequence according to the present invention, the parameters are set, of course, such that the percentage of identity is calculated over the full-length of the reference polypeptide sequence and that gaps in homology of up to 5% of the total number of amino acids in the reference sequence are allowed.

As used herein, the term “altered protein,” “altered polypeptide,” “modified protein,” “modified polypeptide,” “variant polypeptide,” “mutant polypeptide,” “engineered polypeptide,” refers to polypeptides and/or proteins (e.g., an antibody or fragment thereof) comprising at least one amino acid substitution, deletion, and/or addition relative to the native (i.e., wild-type) amino acid sequence, and/or an amino acid substitution that results in altered glycosylation (e.g., hyperglycosylation, hypoglycosylation and/or aglycosylation) at one or more amino acid positions relative to the native, parental (i.e., wild-type) amino acid sequence.

The polypeptide of the present disclosure may also comprise a conservative amino acid substitution. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another embodiment, a string of amino acids can be conservatively replaced with a structurally similar string that differs in order and/or composition of side chain family members.

The polypeptide of the present disclosure may also comprise a supplemental amino acid substitution. A “supplemental amino acid substitution” as used herein is one in which the amino acid residue is replaced with an amino acid that does not alter a specific characteristic of the polypeptide (e.g., an amino acid substitution that does not alter self-interaction).

As used herein, the terms “diffusion interaction parameter” and “ko” may be used interchangeably, except where explicitly stated otherwise. The diffusion interaction parameter/ko is a quantitative measure of protein-protein interactions, including self-interaction (interaction among different molecules of the same type of protein). In some embodiments, the diffusion interaction parameter/ko is measured by dynamic light scattering (DLS) in mL/g.

As used herein, the terms “equilibrium dissociation constant,” “K_{d}” and “binding affinity” may be used interchangeably, except where explicitly stated otherwise. The equilibrium dissociation constant/K_{d} is a quantitative measure of the strength of interactions between two binding partners. Equilibrium dissociation constant/K_{d} is determined by dividing the rate constant k_{off} by the rate constant kₒₙ. In some embodiments, the equilibrium dissociation constant/K_{d} is determined by surface plasmon resonance (SPR) in Moles.

The terms “equilibrium dissociation constant,” “K_{d}” and “binding affinity” are used herein separately and distinctly from the terms “diffusion interaction parameter” and “k_{D}.” Equilibrium dissociation constant is frequently stylized with a capital letter K and a lowercase d in subscript (K_{d}). Conversely, diffusion interaction parameter is frequently stylized with a lowercase letter k with a capital letter D in subscript (k_{D}). Such style is adhered to throughout this application.

As used herein, the term “turbidity” or “opalescence” may be used interchangeably to describe the optical clarity of a solution. The turbidity of protein containing solutions can become less clear as proteins interact with one another. Turbidity is frequently used as an indicator of protein aggregation. As used herein, the term “nephelometric turbidity unit,” “nephelometric turbidity units,” and “NTU” are used interchangeably unless explicitly stated otherwise. NTU is a useful measure of the turbidity, or opalescence, of a solution. In some embodiments, turbidity of a solution is measured in NTU.

As used herein, the term “viscosity” refers to the rate of transfer of momentum in a liquid (Tomar et al. MAbs. 2016; 8(2):216-28). A more viscous solution will flow slower than a less viscous solution. In antibody preparations viscosity is assessed to determine if a certain route of administration may be advantageous or difficult.

Several nomenclatures exist with relation to the numbering of the amino acids within the immunoglobulin constant regions. These include IMGT® (Lefranc et al., Developmental & Comparative Immunology 27:55-77; 2003), EU, and Kabat. Except where noted otherwise, the numbering of the Fc domain is via the EU index as set forth in Kabat (Kabat, Sequences of Proteins of Immunological Interest, National Institutes of Health, Bethesda, MD, 1987 and 1991).

### Polypeptides and Isolated Polypeptides

In one aspect, a polypeptide of the disclosure comprises three amino acid substitutions when compared to a reference sequence of a human IgG4 Fc domain. In some embodiments, a polypeptide of the disclosure has one or more substitutions in the Fc domain compared to a reference sequence of a human IgG4 Fc domain such that the interaction between Fc domains of different polypeptides with an identical sequence is reduced. In some embodiments, a polypeptide of the disclosure has one or more substitutions in the Fc domain compared to a reference sequence of a human IgG4 Fc domain such that the interaction between Fc domains of different polypeptides with an identical sequence is reduced when compared to interactions between different polypeptides with amino acid sequences identical to the reference amino acid sequence.

In some embodiments, a polypeptide of the disclosure comprises an Fc region from human IgG4, wherein the Fc region comprises substitutions at amino acid residues corresponding to amino acid residues Q274, Q355, and E419 according to EU numbering in human IgG4. In some embodiments, a polypeptide of the disclosure comprises a substitution of the glutamine at amino acid residue 274 according to EU numbering in human IgG4 with lysine (Q274K). In some embodiments, a polypeptide of the disclosure comprises a substitution of the glutamine at amino acid residue 355 according to EU numbering in human IgG4 with arginine. In some embodiments, a polypeptide of the disclosure comprises a substitution of the glutamate at amino acid residue 419 according to EU numbering in human IgG4 with glutamine (E419Q). In some embodiments, a polypeptide of the disclosure comprises a substitution of the glutamine at amino acid residue 274 according to EU numbering in human IgG4 with lysine (Q274K) the glutamine at amino acid residue 355 according to EU numbering in human IgG4 with arginine (Q355R), and the glutamate at amino acid residue 419 according to EU numbering in human IgG4 with glutamine (E419Q). In some embodiments, a polypeptide of the disclosure comprises a substitution of the glutamine at amino acid residue 274 according to EU numbering in human IgG4 with lysine (Q274K), a substitution of the glutamine at amino acid residue 355 according to EU numbering in human IgG4 with arginine (Q355R), a substitution of the glutamate at amino acid residue 419 according to EU numbering in human IgG4 with glutamine (E419Q), and a substitution of the glutamate at amino acid residue 294 according to EU numbering in human IgG4 with glutamine (E294Q).

In some embodiments, a polypeptide of the disclosure further comprises a substitution of the amino acid at an amino acid residue corresponding to R409 in human IgG4. In some embodiments, the arginine at amino acid residue 409 according to EU numbering in human IgG4 is substituted with lysine (R409K).

In some embodiments, a polypeptide of the disclosure comprises a substitution of the amino acid at an amino acid residue corresponding to F234 in human IgG4. In some embodiments, the phenylalanine at amino acid residue 234 according to EU numbering in human IgG4 is substituted with alanine (F234A). In some embodiments, a polypeptide of the disclosure comprises a substitution of the amino acid at an amino acid residue corresponding to L235 in human IgG4. In some embodiments, the leucine at amino acid residue 235 according to EU numbering in human IgG4 is substituted with alanine (L235A).

In some embodiments, a polypeptide of the disclosure comprises a substitution of the amino acid at an amino acid residue corresponding to S298 in human IgG4. In some embodiments, the serine at amino acid residue 298 according to EU numbering in human IgG4 is substituted with asparagine (S298N). In some embodiments, a polypeptide of the disclosure comprises a substitution of the amino acid at an amino acid residue corresponding to T299 in human IgG4. In some embodiments, the threonine at amino acid residue 299 according to EU numbering in human IgG4 is substituted with alanine (T299A). In some embodiments, a polypeptide of the disclosure comprises a substitution of the amino acid at an amino acid residue corresponding to Y300 in human IgG4. In some embodiments, the tyrosine at amino acid residue 300 according to EU numbering in human IgG4 is substituted with serine (Y300S). In some embodiments, a polypeptide of the disclosure comprises a substitution of the amino acid at an amino acid residue corresponding to S298 according to EU numbering in human IgG4, T299 according to EU numbering in human IgG4, and Y300 according to EU numbering in human IgG4, with asparagine, alanine, and serine, respectively.

In some embodiments, a polypeptide of the disclosure comprises a substitution of the amino acid at an amino acid residue corresponding to T256 in human IgG4 according to EU numbering. In some embodiments, a polypeptide of the disclosure comprises a substitution of the amino acid at an amino acid residue corresponding to T307 in human IgG4 according to EU numbering. In some embodiments, a polypeptide of the disclosure comprises a substitution at an amino acid residue corresponding to T256 in human IgG4 according to EU numbering and an amino acid residue corresponding to T307 in human IgG4 according to EU numbering. In some embodiments, a polypeptide of the disclosure comprises a substitution of the threonine at amino acid residue 256 according to EU numbering in human IgG4 with aspartic acid (T256D). In some embodiments, a polypeptide of the disclosure comprises a substitution of the threonine at amino acid residue 307 according to EU numbering in human IgG4 with glutamine (T307Q). In some embodiments, a polypeptide of the disclosure comprises a substitution of the threonine at amino acid residue 256 according to EU numbering in human IgG4 with aspartic acid (T256D) and a substitution of the threonine at amino acid residue 307 according to EU numbering in human IgG4 with glutamine (T307Q).

In some embodiments, a polypeptide of the disclosure comprises a substitution of the amino acid at an amino acid residue corresponding to M428 in human IgG4 according to EU numbering. In some embodiments, a polypeptide of the disclosure comprises a substitution of the amino acid at an amino acid residue corresponding to N434 in human IgG4 according to EU numbering. In some embodiments, a polypeptide of the disclosure comprises a substitution at an amino acid residue corresponding to M428 in human IgG4 according to EU numbering and an amino acid residue corresponding to N434 in human IgG4 according to EU numbering. In some embodiments, a polypeptide of the disclosure comprises a substitution of the methionine at amino acid residue 428 according to EU numbering in human IgG4 with leucine (M428L). In some embodiments, a polypeptide of the disclosure comprises a substitution of the asparagine at amino acid residue 434 according to EU numbering in human IgG4 with serine (N434S). In some embodiments, a polypeptide of the disclosure comprises a substitution of the methionine at amino acid residue 428 according to EU numbering in human IgG4 with leucine (M428L) and a substitution of the asparagine at amino acid residue 434 according to EU numbering in human IgG4 with serine (N434S).

In some embodiments, a polypeptide of the disclosure may be further modified through additional amino acid substitutions that do not alter self-interaction. In one aspect, a Fc domain variant polypeptide featured herein has one or more of increased serum half-life, enhanced FcRn binding affinity, enhanced FcRn binding affinity at acidic pH, and/or similar thermal stability, as compared to a Fc domain parent polypeptide.

In some embodiments, a polypeptide of the disclosure comprises one or more self-interaction substitutions. In some embodiments, the polypeptides of the disclosure has a decreased level of self-interaction when compared to a polypeptide with an identical amino acid sequence without the self-interaction substitutions. In some embodiments, a polypeptide of the disclosure has fewer self-interactions as measured by dynamic light scattering compared to an isolated polypeptide comprising the Fc region without said substitutions. In some embodiments, a polypeptide of the disclosure has a decreased diffusion interaction parameter as measured by dynamic light scattering compared to an isolated polypeptide comprising the Fc region without said substitutions. In some embodiments, a polypeptide of the disclosure has a decreased diffusion interaction parameter as measured by dynamic light scattering than the diffusion interaction parameter of an Fc region from a wild-type IgG1 polypeptide. In some embodiments, a polypeptide of the disclosure has a diffusion interaction parameter equal to or greater than 20.0 mL/g when measured by dynamic light scattering. In some embodiments, a polypeptide of the disclosure has a diffusion interaction parameter equal to or greater than 30.0 mL/g when measured by dynamic light scattering. In some embodiments, a polypeptide of the disclosure has a diffusion interaction parameter equal to or greater than 31.8 mL/g when measured by dynamic light scattering. In some embodiments, a polypeptide of the disclosure has a diffusion interaction parameter equal to or greater than 32.0 mL/g when measured by dynamic light scattering. In some embodiments, a polypeptide of the disclosure has a diffusion interaction parameter equal to or greater than 41.0 mL/g when measured by dynamic light scattering. In some embodiments, a polypeptide of the disclosure has a diffusion interaction parameter equal to or greater than 41.5 mL/g when measured by dynamic light scattering. In some embodiments, a polypeptide of the disclosure has a diffusion interaction parameter equal to or greater than 41.8 mL/g when measured by dynamic light scattering.

In some embodiments, a polypeptide comprising one or more self-interaction substitutions does not have a reduced binding affinity to human neonatal Fc receptor (FcRn) when compared to an identical polypeptide lacking the one or more self-interaction substitutions. In some embodiments, a polypeptide comprising one or more self-interaction substitutions does not have a reduced binding affinity to human Fc gamma receptor (FcγR) when compared to an identical polypeptide lacking the one or more self-interaction substitutions. In some embodiments, a polypeptide comprising one or more self-interaction substitutions does not have an increased binding affinity to human Fc gamma receptor (FcγR) when compared to an identical polypeptide lacking the one or more self-interaction substitutions. In some embodiments, a polypeptide of the disclosure has a binding affinity (K_{d}) for human FcRn of 1.22E−06 M. In some embodiments, a polypeptide of the disclosure has a binding affinity (K_{d}) for human FcRn of 1.19E−06 M. In some embodiments, a polypeptide of the disclosure has a binding affinity (K_{d}) for human FcRn of 1.18E−06 M.

In some embodiments, a composition or solution comprising the polypeptides comprising one or more self-interaction substitutions is less viscous than a solution comprising a polypeptide with an identical amino acid sequence without the self-interaction substitutions. In some embodiments, the viscosity of the composition or solution is measured in centiPoise (cP). In some embodiments, the composition or solution comprising the polypeptide comprising one or more self-interaction substitutions has a viscosity of less than 20 cP. In some embodiments, the composition or solution comprising the polypeptide comprising one or more self-interaction substitutions has a viscosity of less than 15 cP. In some embodiments, the composition or solution comprising the polypeptide comprising one or more self-interaction substitutions has a viscosity of less than 13.9 cP. In some embodiments, the composition or solution comprising the polypeptide comprising one or more self-interaction substitutions has a viscosity of less than 12.2 cP. In some embodiments, the composition or solution comprising the polypeptide comprising one or more self-interaction substitutions has a viscosity of less than 8.5 cP. In some embodiments, the composition or solution comprising the polypeptide comprising one or more self-interaction substitutions has a viscosity of less than 7.9 cP.

In some embodiments, a composition or solution comprising the polypeptides comprising one or more self-interaction substitutions is less turbid than a solution comprising a polypeptide with an identical amino acid sequence without the self-interaction substitutions. In some embodiments, the turbidity of the composition or solution is measured by determining opalescence in nephelometric turbidity units (NTU). In some embodiments, the composition or solution comprising the polypeptide comprising one or more self-interaction substitutions has an opalescence of less than or equal to 12 NTU. In some embodiments, the composition or solution comprising the polypeptide comprising one or more self-interaction substitutions has an opalescence of less than or equal to 8.7 NTU. In some embodiments, the composition or solution comprising the polypeptide comprising one or more self-interaction substitutions has an opalescence of less than or equal to 7.3 NTU. In some embodiments, the composition or solution comprising the polypeptide comprising one or more self-interaction substitutions has an opalescence of less than or equal to 6.7 NTU.

In one aspect, the present disclosure provides an isolated Fc domain variant comprising or complexed with (e.g., fused to) at least one binding domain (e.g., at least one binding polypeptide). In certain embodiments, the binding domain comprises one or more antigen binding domains. The antigen binding domains need not be derived from the same molecule as the parental Fc domain. In certain embodiments, the Fc domain variant is present in an antibody.

In one embodiment, an Fc domain variant is present in an antibody or is complexed with an antibody. Any antibody from any source or species can be employed with an Fc domain variant disclosed herein. Suitable antibodies include without limitation, chimeric antibodies, humanized antibodies, or human antibodies. Suitable antibodies also include without limitation, full-length antibodies, monoclonal antibodies, polyclonal antibodies, or single-domain antibodies, such as VHH antibodies.

In certain exemplary embodiments, an Fc domain variant may be bound to or complexed with an antigen-binding fragment of an antibody. The term “antigen-binding fragment” refers to a polypeptide fragment of an immunoglobulin or antibody which binds antigen or competes with intact antibody (i.e., with the intact antibody from which they were derived) for antigen binding (i.e., specific binding). Antigen-binding fragments can be produced by recombinant or biochemical methods that are well known in the art. Exemplary antigen-binding fragments include Fv, Fab, Fab′, and (Fab′)2. In certain exemplary embodiments, a polypeptide of the current disclosure comprises at least one antigen-binding fragment and an Fc domain variant.

In some embodiments, the polypeptide comprises a single chain variable region sequence (ScFv). Single chain variable region sequences comprise a single polypeptide having one or more antigen binding sites, e.g., a VL domain linked by a flexible linker to a VH domain. ScFv molecules can be constructed in a VH-linker-VL orientation or VL-linker-VH orientation. The flexible hinge that links the VL and VH domains that make up the antigen binding site includes from about 10 to about 50 amino acid residues. Connecting peptides are known in the art. Binding polypeptides comprises at least one scFv and/or at least one constant region. In one embodiment, a binding polypeptide of the current disclosure comprises at least one scFv linked or fused to an Fc domain variant.

In some embodiments, a polypeptide of the current disclosure is a multivalent (e.g., tetravalent) antibody which is produced by fusing a DNA sequence encoding an antibody with a ScFv molecule (e.g., an altered ScFv molecule). For example, in one embodiment, these sequences are combined such that the ScFv molecule (e.g., an altered ScFv molecule) is linked at its N-terminus or C-terminus to an Fc domain variant via a flexible linker (e.g., a gly/ser linker). In another embodiment a tetravalent antibody of the current disclosure can be made by fusing an ScFv molecule to a connecting peptide, which is fused to an Fc domain variant to construct an ScFv-Fab tetravalent molecule.

In another embodiment, a polypeptide of the current disclosure is an altered minibody. An altered minibody of the current disclosure is a dimeric molecule made up of two polypeptide chains each comprising an ScFv molecule which is fused to an Fc domain variant via a connecting peptide. Minibodies can be made by constructing an ScFv component and connecting peptide components using methods described in the art (see, e.g., U.S. Pat. No. 5,837,821 or WO 94/09817A1). In another embodiment, a tetravalent minibody can be constructed. Tetravalent minibodies can be constructed in the same manner as minibodies, except that two ScFv molecules are linked using a flexible linker. The linked scFv-scFv construct is then joined to an Fc domain variant.

In another embodiment, a polypeptide of the current disclosure comprises a diabody. Diabodies are dimeric, tetravalent molecules each having a polypeptide similar to scFv molecules, but usually having a short (less than 10, e.g., about 1 to about 5) amino acid residue linker connecting both variable domains, such that the VL and VH domains on the same polypeptide chain cannot interact. Instead, the VL and VH domain of one polypeptide chain interact with the VH and VL domain (respectively) on a second polypeptide chain (see, for example, WO 02/02781). Diabodies of the current disclosure comprise an scFv-like molecule fused to an Fc domain variant.

In another embodiment, a polypeptide of the current disclosure comprises a single-domain antibody (sdAb), also referred to as a VHH or a nanobody. Nanobody® is registered trademark of Ablynx. VHHs comprise variable heavy chain domains devoid of light chains. Similar to conventional VH domains, VHHs contain four FRs and three CDRs. VHHs have advantages over conventional antibodies. As they are about ten times smaller than full-size IgG molecules, properly folded functional VHHs can be produced by in vitro expression while achieving high yield. Furthermore, VHHs are very stable, and resistant to the action of proteases. The properties and production of VHHs have been reviewed by Harmsen and De Haard H J (Appl. Microbiol. Biotechnol. 2007 November; 77(1):13-22). In certain exemplary embodiments, an Fc domain variant of IgG4 is fused with one or more VHHs.

In other embodiments, polypeptides of the disclosure comprise multi-specific or multivalent antibodies comprising one or more variable domain in series on the same polypeptide chain, e.g., tandem variable domain (TVD) polypeptides. Exemplary TVD polypeptides include the “double head” or “Dual-Fv” configuration described in U.S. Pat. No. 5,989,830. In the Dual-Fv configuration, the variable domains of two different antibodies are expressed in a tandem orientation on two separate chains (one heavy chain and one light chain), wherein one polypeptide chain has two VH domains in series separated by a peptide linker (VH1-linker-VH2) and the other polypeptide chain consists of complementary VL domains connected in series by a peptide linker (VL1-linker-VL2). In the cross-over double head configuration, the variable domains of two different antibodies are expressed in a tandem orientation on two separate polypeptide chains (one heavy chain and one light chain), wherein one polypeptide chain has two VH domains in series separated by a peptide linker (VH1-linker-VH2) and the other polypeptide chain consists of complementary VL domains connected in series by a peptide linker in the opposite orientation (VL2-linker-VL1). Additional antibody variants based on the “Dual-Fv” format include the Dual-Variable-Domain IgG (DVD-IgG) bispecific antibody (see U.S. Pat. No. 7,612,181 and the TBTI format (see US 2010/0226923 A1). In some embodiments, polypeptides of the disclosure comprise multi-specific or multivalent antibodies comprising one or more variable domain in series on the same polypeptide chain fused to an Fc domain variant.

In another embodiment, a polypeptide of the disclosure comprises a cross-over dual variable domain IgG (CODV-IgG) bispecific antibody based on a “double head” configuration (see US20120251541 A1, which is incorporated by reference herein in its entirety).

In other embodiments, a polypeptide of the disclosure comprises a CrossMab or a CrossMab-Fab multispecific format (see WO2009080253 and Schaefer, et al., PNAS (2011), 108: 11187-1191). Antibody variants based on the CrossMab format have a crossover of antibody domains within one arm of a bispecific IgG antibody enabling correct chain association.

A polypeptide of the present disclosure, comprising an Fc domain variant described herein, can include the CDR sequences or the variable domain sequences of a known “parent” antibody. In some embodiments, the parent antibody and the antibody of the disclosure can share similar or identical sequences except for modifications to the Fc domain as disclosed herein.

In another embodiment, a polypeptide of the disclosure comprises a therapeutic polypeptide. In some embodiments, the therapeutic polypeptide may be a receptor, a ligand, or an enzyme. In some embodiments, the therapeutic polypeptide may be a growth factor. The growth factor can be selected from any growth factor known in the art. In some embodiments, the growth factor is a hormone, in other embodiments, the growth factor is a cytokine. In some embodiments, the growth factor is a chemokine. In some embodiments, the polypeptide comprises a therapeutic molecule or therapeutic polypeptide linked to the N-terminus and/or the C-terminus of the Fc domain of the present invention. In some embodiments, the polypeptide is an Fc-fusion polypeptide.

Alternatively, or additionally, in some embodiments, the polypeptides provided herein undergo co- and post-translational modifications as known in the art. Examples of post-translational modifications include, but are not limited to, disulfide bond formation, glycosylation, cyclization (such as e.g., N-terminal pyroglutamate formation), have a N-terminal or C-terminal residue removed or “clipped” (for example, C-terminal lysine residues are often removed during the manufacturing process), deamidation, isomerization, oxidation, glycation, acylation, fucosylation, peptide bond cleavage, non-reductible cross-linking, truncation, and/or have part or all of a signal sequence incompletely processed.

In another embodiment, a polypeptide of the disclosure further comprises a lysine at the C-terminal end of the polypeptide. In some embodiments, a polypeptide of the disclosure further comprises a lysine at the C-terminal end of the Fc region. In some embodiments, the light chain and/or heavy chain comprises an N-terminal and/or C-terminal truncation of 1, 2, 3, 4, or 5 amino acids.

### Nucleic Acids

In one aspect, polynucleotides encoding the Fc domain variants and/or the binding polypeptides disclosed herein are provided. Methods of making an Fc domain variant and/or a binding polypeptide comprising expressing these polynucleotides are also provided.

Polynucleotides encoding the Fc domain variants and/or the binding polypeptides disclosed herein are typically inserted in an expression vector for introduction into host cells that may be used to produce the desired quantity of the claimed polypeptides. Accordingly, in certain aspects, the invention provides expression vectors comprising polynucleotides disclosed herein and host cells comprising these vectors and polynucleotides.

The term “vector” or “expression vector” is used herein for the purposes of the specification and claims, to mean vectors used for introducing into and expressing a desired gene in a cell. As known to those skilled in the art, such vectors may easily be selected from the group consisting of plasmids, phages, viruses and retroviruses. In general, a vector will comprise a selection marker, appropriate restriction sites to facilitate cloning of the desired gene and the ability to enter and/or replicate in eukaryotic or prokaryotic cells.

Numerous expression vector systems may be employed. For example, one class of vector utilizes DNA elements which are derived from animal viruses such as bovine papilloma virus, polyoma virus, adenovirus, vaccinia virus, baculovirus, retroviruses (RSV, MMTV or MOMLV), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. In some embodiments the cloned variable region genes are inserted into an expression vector along with the heavy and light chain constant region genes (such as human genes) synthesized as discussed above.

In other embodiments, a polypeptide as described herein may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980 which is incorporated by reference herein. Those skilled in the art will appreciate that such expression systems may be used to effectively produce the full range of polypeptides disclosed in the instant application.

More generally, once a vector or DNA sequence encoding an Fc domain variant and/or a polypeptide of the present disclosure has been prepared, the expression vector may be introduced into an appropriate host cell. That is, the host cell may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, e.g., Ridgway, A. A. G. “Mammalian Expression Vectors” Chapter 24.2, pp. 470-472 Vectors, Rodriguez and Denhardt, Eds. (Butterworths, Boston, MA 1988). The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype and consequently results in a change in the recipient cell.

Along those same lines, “host cells” refer to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, or from the cell culture containing both the medium and the suspended cells.

In one embodiment, the host cell line used for expression of a polypeptide is of eukaryotic or prokaryotic origin. In one embodiment, the host cell line used for expression of a polypeptide is of bacterial origin. In one embodiment, the host cell line used for expression of a polypeptide is of mammalian origin. Those skilled in the art can determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), BFA-1c1BPT (bovine endothelial cells), RAJI (human lymphocyte), 293 (human kidney). In one embodiment, the cell line provides for altered glycosylation, e.g., afucosylation, of the antibody expressed therefrom (e.g., PER.C6® (Crucell) or FUT8-knock-out CHO cell lines (POTELLIGENT™ cells) (Biowa, Princeton, NJ)). In one embodiment NS0 cells may be used. Host cell lines are typically available from commercial services, the American Tissue Culture Collection or from published literature.

In vitro production allows scale-up to give large amounts of the desired polypeptide. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

One or more genes encoding polypeptides can also be expressed in non-mammalian cells such as bacteria or yeast or plant cells. In this regard, it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the polypeptides can become part of inclusion bodies. The polypeptides must be isolated, purified and then assembled into functional molecules.

In addition to prokaryotes, eukaryotic microbes may also be used. Saccharomyces cerevisiae, or common baker's yeast, is the most commonly used among eukaryotic microorganisms although a number of other strains are commonly available. For expression in Saccharomyces, the plasmid YRp7, for example, (Stinchcomb et al., Nature, 282:39 (1979); Kingsman et al., Gene, 7:141 (1979); Tschemper et al., Gene, 10:157 (1980)) is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trp1 lesion as a characteristic of the yeast host cell genome then provides an effective environment for detecting transformation by growth in the absence of tryptophan.

The invention also includes a method of making a polypeptide of the disclosure, comprising transfecting one or more host cell with a polynucleotide or vector encoding the polypeptide and expressing the polypeptide in the host cell.

### Methods of the Disclosure

In one aspect, the invention provides methods of treating a disease or disorder in a subject in need thereof comprising administering to the subject an effective amount of an Fc domain variant disclosed herein. In certain embodiments, the present disclosure provides kits and methods for the treatment of diseases and disorders, e.g., cancer, in a mammalian subject in need of such treatment.

In another embodiment, the subject Fc domain variants are useful for the treatment of disorders, including, without limitation, infectious diseases, autoimmune disorders, inflammatory disorders, lung diseases, neuronal or neurodegenerative diseases, liver diseases, diseases of the spine, diseases of the uterus, depressive disorders and the like. Non-limiting examples of infectious diseases include those caused by RNA viruses (e.g., orthomyxoviruses (e.g., influenza), paramyxoviruses (e.g., respiratory syncytial virus, parainfluenza virus, metapneumovirus), rhabdoviruses (e.g., rabies virus), coronaviruses (e.g., SARS-CoV), alphaviruses (e.g., Chikungunya virus) lentiviruses (e.g., HIV) and the like) or DNA viruses. Examples of infectious diseases also include, without limitation, bacterial infectious diseases, caused by, e.g., Staphylococcus aureus, Staphylococcus epidermidis, Enterococcus, Streptococcus, Escherichia coli, and other infectious diseases including, e.g., those caused by Candida albicans. Other infectious diseases include, without limitation, malaria, SARS, yellow fever, Lyme borreliosis, leishmaniasis, anthrax and meningitis. Exemplary autoimmune disorders include, but are not limited to, psoriasis and lupus.

### Compositions

In one aspect, compositions comprising the polypeptides of the disclosure are provided. Compositions comprising the polypeptides of the present disclosure may contain a suitable pharmaceutically acceptable carrier. For example, they may contain excipients that allow or aid the polypeptides to be prepared for delivery to a subject. In one aspect, the compositions are pharmaceutical compositions. Pharmaceutical compositions of the disclosure can be prepared such that they are suitable for parenteral administration (i.e. intravenous, subcutaneous, or intramuscular). The pharmaceutical compositions may be in the form of a suspension, a solution, or an emulsion. Alternatively, the composition can be in powder form for constitution with a suitable vehicle, e.g., pyrogen free water.

In some embodiments, the composition is administered by topical administration. In some embodiments, the composition is administered by intraocular administration. In some embodiments, the composition is administered by parenteral administration. In some embodiments, the composition is administered by intrathecal administration. In some embodiments, the composition is administered by subdural administration. In some embodiments, the composition is administered by oral administration. The parenteral administration can be intravenous or subcutaneous administration.

Compositions of the invention may be in a variety of forms, including, for example, liquid (e.g., injectable and infusible solutions), dispersions, suspensions, semi-solid and solid forms. The preferred form depends on the mode of administration and therapeutic application. Additional active ingredients may be incorporated into the compositions.

In addition to polypeptides, a liquid dosage form may contain inert ingredients such as water, ethyl alcohol, ethyl carbonate, ethyl acetate, benzyl alcohol, benzyl benzoate, propylene glycol, 1 3-butylene glycol, dimethylformamide, oils, glycerol, tetrahydrofurfuryl alcohol, polyethylene glycols, and fatty acid esters of sorbitan.

Examples of suitable pharmaceutical carriers are also described in Remington's Pharmaceutical Sciences by E. W. Martin. Some non-limiting examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition may also contain pH buffering reagents, and wetting or emulsifying agents.

### Additional Embodiments

In an embodiment, a polypeptide of the disclosure comprises the amino acid sequence of SEQ ID NO: 5. In an embodiment, a polypeptide of the disclosure comprises the amino acid sequence of SEQ ID NO: 6. In an embodiment, a polypeptide of the disclosure comprises the amino acid sequence of SEQ ID NO: 7. In an embodiment, a polypeptide of the disclosure comprises the amino acid sequence of SEQ ID NO: 8. In an embodiment, a polypeptide of the disclosure comprises the amino acid sequence of SEQ ID NO: 12. In an embodiment, a polypeptide of the disclosure comprises the amino acid sequence of SEQ ID NO: 13. In an embodiment, a polypeptide of the disclosure comprises the amino acid sequence of SEQ ID NO: 14. In an embodiment, a polypeptide of the disclosure comprises the amino acid sequence of SEQ ID NO: 15. In an embodiment, a polypeptide of the disclosure comprises the amino acid sequence of SEQ ID NO: 16. In an embodiment, a polypeptide of the disclosure comprises the amino acid sequence of SEQ ID NO: 17. In an embodiment, a polypeptide of the disclosure comprises the amino acid sequence of SEQ ID NO: 20. In an embodiment, a polypeptide of the disclosure comprises the amino acid sequence of SEQ ID NO: 21. In an embodiment, a polypeptide of the disclosure comprises the amino acid sequence of SEQ ID NO: 22. In an embodiment, a polypeptide of the disclosure comprises the amino acid sequence of SEQ ID NO: 25. In an embodiment, a polypeptide of the disclosure comprises the amino acid sequence of SEQ ID NO: 26. In an embodiment, a polypeptide of the disclosure comprises the amino acid sequence of SEQ ID NO: 27. In an embodiment, a polypeptide of the disclosure comprises the amino acid sequence of SEQ ID NO: 28. In an embodiment, a polypeptide of the disclosure comprises the amino acid sequence of SEQ ID NO: 31. In an embodiment, a polypeptide of the disclosure comprises the amino acid sequence of SEQ ID NO: 32. In an embodiment, a polypeptide of the disclosure comprises the amino acid sequence of SEQ ID NO: 33. In an embodiment, a polypeptide of the disclosure comprises the amino acid sequence of SEQ ID NO: 34.

Having now described the present disclosure in detail, the same will be more clearly understood by reference to the following examples. The following examples are included solely for purposes of illustration and are not considered limiting embodiments. All patents and publications referred to herein are expressly incorporated by reference.

### EXAMPLES

Throughout these examples, except where otherwise specified, polypeptides were prepared as follows: polypeptides were dialyzed into 10 mM histidine pH 6.0 buffer and concentrated to a target concentration of 140 mg/mL. The concentrations were checked using a Lunatic—UVNis Spectrophotomer (UNchained Labs). Samples were filtered using 0.22 μm microcentrifuge filters (Millipore) where material was sufficient. Polypeptides of the examples used consistent binding regions within experiments. Examples 1-3 were performed using the binding region of BR-A. Various other binding regions are appended to IgG Fc or constant regions throughout the examples; BR-A, BR-B, BR-C, BR-D, and BR-E are binding regions that each target a distinct antigen.

### Example 1: Introduction of Amino Acid Substitutions into IgG4 to Analyze High Concentration Fc Interactions

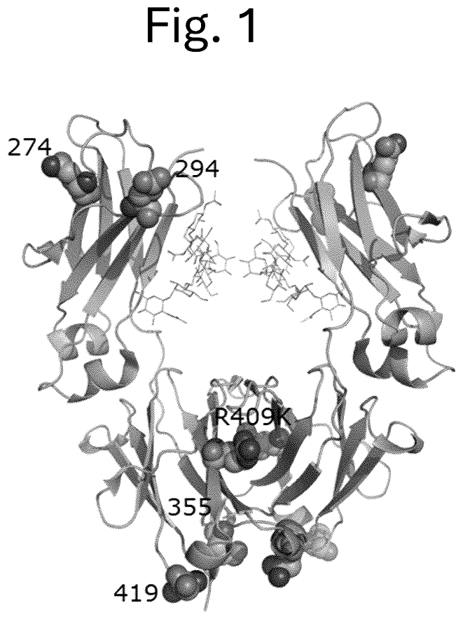

IgG4 antibodies are known to have increased self-interaction and aggregation when compared to IgG1 antibodies. However, IgG4 may be a preferable format where effector function is not a desirable attribute. To determine if self-interaction and aggregation could be reduced in IgG4 molecules, amino acid substitutions were introduced into polypeptides comprising the Fc region of the human IgG4_PE sequence (SEQ ID NO: 18; S228P/L235E). The S228P/L235E substitutions in the IgG4 Fc region are known stabilize the hinge region of IgG4 (S228P) and decrease FcγR binding. Positions Q274, Q355, and E419 are surface exposed in IgG4 Fc (FIG. 1) and were substituted with the amino acid residues present in the corresponding positions in IgG1 Fc. Position E294 is also surface exposed (FIG. 1) and negatively charged, and was therefore substituted a neutral residue (E294Q). IgG4 is also known to aggregate at low pH. This effect is known to be mitigated by substitution of the arginine at position 409 with lysine (R409K). Polypeptides with the R409K substitution were also examined with the Q274/E294/Q355/E419 substitutions. A consistent binding region was used for the substitution polypeptides.

Diffusion Interaction Parameter (ko), a measure of protein self-interaction, was assessed by dynamic light scattering (DLS) Analysis of ko-DLS (diffusion interaction parameter by dynamic light scattering) was performed using a Wyatt DynaPro Plate Reader II dynamic light scattering instrument. The antibodies were dialyzed into 10 mM histidine pH 6.0 and concentrated to 5 mg/mL. Dilutions of the stock solution from 4.5-1 mg/mL in 0.5 mg/mL increments were made in triplicate. The samples were vacuum filtered using a 0.45 um filter plate (MilliPore). The filtrate was collected in a Greiner Bio-One UV-STAR 384-well plate. The apparent diffusion coefficient (Dₐₚₚ) was measured in each well at 20° C. and the concentration was measured using a Stunner UV-Vis and dynamic light scattering instrument (UNchained Labs). DO is the apparent diffusion coefficient at 0 mg/mL. Each Dₐₚₚ was plotted as a function of concentration and a linear fit was performed to determine the diffusion interaction parameter (ko, in mL/g) as previously described (Kingsbury et al., Sci. Adv. 2020). Results are displayed in Table 1.

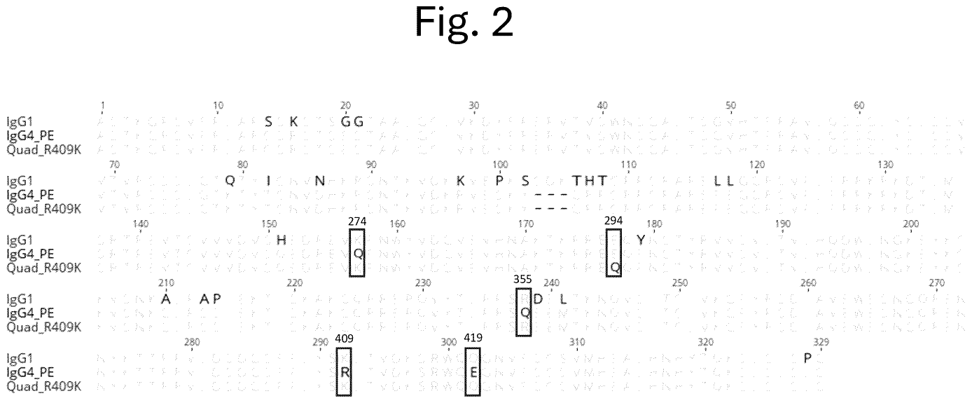

13 different substitution combinations were created, including reverting Q274, Q355, and E419 to the corresponding residue in IgG1 (FIG. 2), while the negatively charged E294 was substituted with a neutral residue (E294Q). Protein self-interaction for these 13 IgG4 substitutions as well as human IgG1 were assessed by ko-DLS. Results are shown in Table 1. As expected, human IgG1 exhibited a large positive ko value, indicating minimal protein self-interaction. Samples in which the IgG4 Fc region only comprised S228P/L235E substitutions, or S228P/L235E/R409K substitutions, exhibited a negative kD value, indicating a greater level of protein self-interaction. When the Q274K, E294Q, Q355R, and E419Q substitutions were tested individually in the IgG4_PE Fc region, each exhibited modest positive ko values (huIgG4_PE_Single_1, huIgG4_PE_Single_2, huIgG4_PE_Single_3, and huIgG4_PE_Single_4). Further improvement in self-interaction characteristics were seen in samples in which Q274K, Q355R, and E419Q were tested in combinations of two substitutions in the IgG4_PE Fc region (huIgG4_PE_Double_1, huIgG4_PE_Double_2, and huIgG4_PE_Double_3). When Q274K, Q355R, and E419Q substitutions were incorporated into the IgG4_PE Fc region (huIgG4_PE_Triple), ko values were further increased. This effect was not substantially altered when R409K was introduced (huIgG4_PE_Triple_R409K). Finally, the IgG4_PE Fc region was mutated to incorporate the substitutions Q274K/E294Q/Q355R/E419Q (huIgG4_PE_Quad). The inclusion of all four substitutions still further limited self-interaction. As with huIgG4_PE_Triple, the substitution of arginine 409 with lysine did not significantly alter this effect.

TABLE 1
Diffusion Interaction Parameter Quantification
of Single and Multiple IgG4 Substitutions

| | Fc substitutions from Reference | k_{D} |
|---|---|---|
| Sample | Sequence | (mL/g) |
| huIgG1 | No substitutions - SEQ ID NO: 1 | 52.9 |
| huIgG4_PE | S228P/L235E | −6.2 |
| huIgG4_PE_R409K | S228P/L235E/R409K | −9.9 |
| huIgG4_PE_Single_1 | S228P/L235E/Q274K | 4.1 |
| huIgG4_PE_Single_2 | S228P/L235E/E294Q | 10 |
| huIgG4_PE_Single_3 | S228P/L235E/Q355R | 2.8 |
| huIgG4_PE_Single_4 | S228P/L235E/E419Q | 15.4 |
| huIgG4_PE_Double_1 | S228P/L235E/Q355R/E419Q | 25.4 |
| huIgG4_PE_Double_2 | S228P/L235E/Q274K/E419Q | 22.4 |
| huIgG4_PE_Double_3 | S228P/L235E/Q274K/Q355R | 27.4 |
| huIgG4_PE_Triple | S228P/L235E/Q274K/Q355R/ | 32 |
| | E419Q | |
| huIgG4_PE_Triple_{—} | S228P/L235E/Q274K/Q355R/ | 31.8 |
| R409K | E419Q/R409K | |
| huIgG4_PE_Quad | S228P/L235E/Q274K/E294Q/ | 41.5 |
| | Q355R/E419Q | |
| huIgG4_PE_Quad_{—} | S228P/L235E/Q274K/E294Q/ | 41.8 |
| R409K | Q355R/E419Q/R409K | |

### Example 2: Further Analysis of Amino Acid Substitutions on High Concentration Characteristics of IgG4 Fc Regions

In addition to self-interaction as determined by DLS, viscosity and opalescence, indicators of quality for antibody therapeutics, were also analyzed. The binding region of the BR-A was appended to a polypeptide with an IgG1 Fc region, an IgG4_PE Fc region, or IgG4_PE Fc regions with substitutions to determine if IgG4 viscosity and opalescence could be engineered to exhibit IgG1-like levels. Analysis of viscosity of antibodies was performed using a VROC Initium viscometer (RheoSense). A single point viscosity measurement was collected on the antibodies at 20° C. Analysis of μ-nephelometry was performed using a Wyatt DynaPro NanoStar laser light scattering instrument. The laser power of the instrument was decreased to 1% and the static scattering detector voltage was recorded and calibrated against a set of polymer bead turbidity standards ranging from 1 to 100 NTU (Millipore Sigma). Results are shown in Table 2.

Human IgG1 exhibited low viscosity (8.4 centiPoise, cP) and low opalescence (9.2 nephelometric turbidity units, NTU). By contrast, the IgG4_PE and IgG4_PE_R409K Fc regions exhibited high viscosity (23.6 cP and 30 cP, respectively) and high opalescence (14 NTU and 14.9 NTU, respectively). When the triple substitution of Q274K/Q355R/E419Q was engineered into the IgG4_PE Fc region, viscosity was lowered from the IgG4_PE Fc region, and opalescence was lower than that of human IgG1. Addition of the R409K substitution to the triple substitution exhibited similar characteristics. Finally, the quadruple substitution Q274K/E294Q/Q355R/E419Q, with or without an additional R409K substitution, exhibited lower viscosity than human IgG1. The quadruple substitution with the additional R409K substitution also exhibited decreased opalescence compared to human IgG1. The quadruple substitution alone exhibited an increased opalescence when compared to the human IgG1 or IgG4_PE Fc region, potentially due to impurity of the sample used. This increased opalescence was reversed when a purer sample was obtained and tested.

TABLE 2
Viscosity and Opalescence Analysis of IgG4 Variants

| | Viscosity (cP) @ | Opalescence (NTU) |
|---|---|---|
| Sample | 140 mg/mL | @ 140 mg/mL |
| huIgG1 | 8.4 | 9.2 |
| huIgG4_PE | 23.6 | 14 |
| huIgG4_PE_R409K | 30 | 14.9 |
| huIgG4_PE_Triple | 13.9 | 7.3 |
| huIgG4_PE_Triple_R409K | 12.2 | 8.7 |
| huIgG4_PE_Quad | 8.5 | 16.4/10.9* |
| huIgG4_PE_Quad_409K | 7.9 | 6.7 |

*purer sample

### Example 3: Fc Receptor Binding to IgG4 Fc Regions with Amino Acid Substitutions

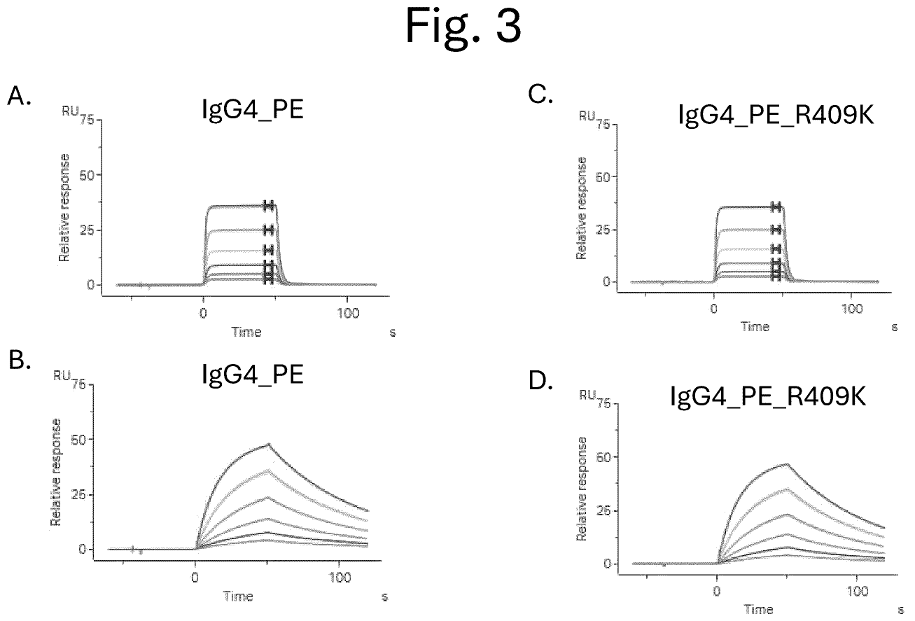

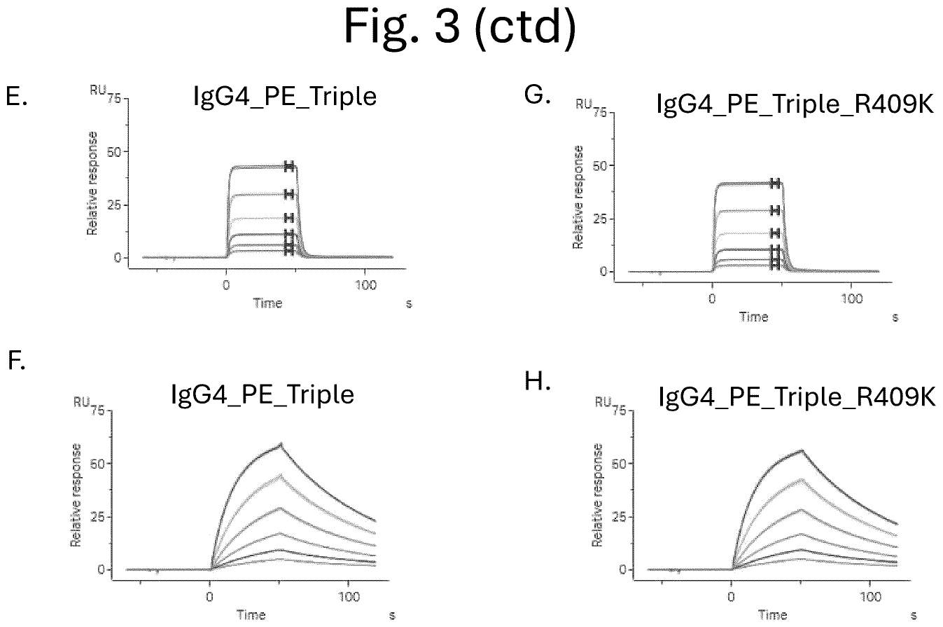

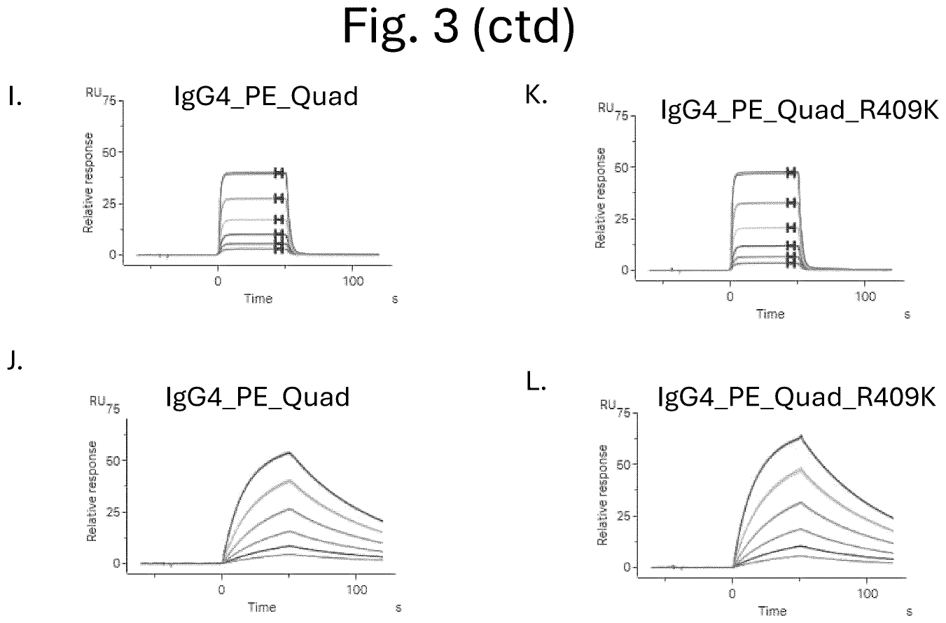

The characteristics of the self-interaction substitutions (IgG4_PE_Triple, Q274K/Q355R/E419Q; IgG4_PE_Quad, Q274K/E294Q/Q355R/E419Q) were compared to an IgG4 Fc region with only the S228P/L235E substitutions (IgG4_PE) and S228P/L235E/R409K (IgG4_PE_R409K) for their ability to bind both the human neonatal Fc receptor (FcRn) and the mouse FcRn (FIG. 3), using a consistent binding region (BR-A). Binding was analyzed by surface plasmon resonance (SPR) using a Biacore 8K instrument. CaptureSelect™ human Fab kappa biotin conjugate was captured to a CM5 series S chip immobilized with streptavidin to achieve a surface density of ˜1500 relative response units (RU). Antibodies were diluted to 2 μg/mL in PBS-T pH 5.8 running buffer and captured to anti-Fab at ˜120 RU. Recombinant human FcRn was serially diluted 2-fold from 1.5 μM in running buffer for a total of 6 concentrations. Mouse FcRn was similarly diluted from 1 μM. Receptors were injected in the flow over capture antibody for 50 sec at 25° C. followed by 60 sec dissociation. The surface was regenerated with 10 mM glycine pH 1.5 for 60 sec and 10 mM NaOH for 60 sec at 50 μL/min flowrate. Sensorgrams were processed using the Biacore Insight software. A report point was taken at equilibrium for each injection and plotted against the human FcRn concentration and affinity was calculated using a non-linear curve fit (steady state analysis) or with a 1:1 kinetic binding model for mouse FcRn. Results are shown in sensorgram (FIG. 3), with equilibrium dissociation constant (K_{d}) shown in Table 3. No significant difference was seen between binding of IgG4_PE and either the IgG4_PE_Triple or IgG4_PE_Quad substitution Fc region to either human FcRn (FIGS. 3A, 3E, and 3I, respectively) or mouse FcRn (FIGS. 3B, 3F, and 3J, respectively). Additionally, no significant difference was seen between binding of IgG4_PE_R409K and either the IgG4_PE_Triple_R409K or IgG4_PE_Quad_R409K substitution Fc region to either human FcRn (FIGS. 3C, 3G, and 3K, respectively) or mouse FcRn (FIGS. 3D, 3H, and 3L, respectively).

TABLE 3
FcRn Binding Characteristics of IgG4 Variants

| | Human FcRn | Mouse FcRn |
|---|---|---|
| Sample | Steady State K_{d} (M) | 1:1 Binding K_{d} (M) |
| IgG1 | 1.00E−06 | 2.04E−07 |
| IgG4_PE_Quad | 1.22E−06 | 2.43E−07 |
| IgG4_PE_Triple | 1.18E−06 | 2.30E−07 |
| IgG4_PE | 1.17E−06 | 2.43E−07 |
| IgG4_PE_Quad_R409K | 1.19E−06 | 2.34E−07 |
| IgG4_PE_Triple_R409K | 1.18E−06 | 2.28E−07 |
| IgG4_PE_R409K | 1.14E−06 | 2.45E−07 |

Binding of IgG4 variants to FcγR was also analyzed by SPR. The anti-human Fab capture method described for FcRn binding was used except for the following differences. Antibodies and receptors were diluted in HBS-EP+ (10 mM HEPES pH 7.4, 150 mM NaCl, 3 mM EDTA, 0.05% surfactant P20). Human FcγRI was serially diluted 2-fold from 12.5 nM for a total of 6 concentrations and injected for 180 sec with 600 sec dissociation. The low affinity receptors were diluted from 8, 18, 6, 6, 1.5, and 1.0 for FcγRIIa, FcγRIIb, FcγRIIIaV, and FcγRIIIaF, respectively. The surface was regenerated as described previously. Steady state analysis was used for all low affinity receptors, while a 1:1 kinetic binding model fit FcγRI sensorgrams to obtain a K_{d}. As the S228P/L235E substitutions are known to abrogate FcγR binding, the IgG4_PE_Triple and IgG4_PE_Quad substitutions were tested to determine if they altered this known phenomenon, with and without the additional R409K substitution. Results are shown in Table 4. None of the IgG4 Fc region with substitutions showed a significant increase in FcγR binding affinity compared to IgG4_PE.

TABLE 4
FcyR Binding Characteristics of IgG4 Variants

| | | hCD32a | | hCD16a | hCD16a |
|---|---|---|---|---|---|
| | hCD64 | (R167) | hCD32b | (V158) | (F158) |
| | (FcγRIa) | (FcγRIIa) | (FcγRIIb) | (FcγRIIIa) | (FcγRIIIa) |
| Sample | K_{d} (M) | K_{d} (M) | K_{d} (M) | K_{d} (M) | K_{d} (M) |
| IgG1 | 8.36E−11 | 1.13E−06 | 3.96E−06 | n.d. | 2.43E−06 |
| IgG4_PE | n.d. | 5.85E−06 | 8.91E−06 | n.d. | n.d. |
| IgG4_PE_Quad | n.d. | >8.00E−06* | 9.15E−06 | n.d. | n.d. |
| IgG4_PE_Triple | n.d. | 7.20E−06 | 5.23E−06 | n.d. | n.d. |
| IgG4_PE_Quad_R409K | n.d. | >8.00E−06* | 4.62E−06 | n.d. | n.d. |
| IgG1 | 8.36E−11 | 1.13E−06 | 3.96E−06 | n.d. | 2.43E−06 |

n.d.—not determined (negligible binding)
*Concentration range insufficient for accurate affinity calculation

### Example 4: Analysis of Self-Interaction Substitutions with Different Binding Regions

The improved characteristics seen from the self-interaction substitutions when compared to the IgG4_PE Fc region were analyzed in the context of being appended to different binding regions. Examples 1-3 were performed using polypeptides with the binding region of BR-A. In this example, additional binding regions were appended to polypeptides with varying Fc regions—IgG1 (SEQ ID NO: 1), IgG4_PE, IgG4_PE_Triple, IgG4_PE_Triple_R409K, IgG4_PE_Quad, and IgG4_PE_Quad_R409K. Samples were then analyzed by ko-DLS as previously described. Results are seen in Table 5.

All tested proteins exhibited low self-interaction. The IgG4_PE Fc region increased protein self-interaction regardless of binding region, as can be seen by low positive and negative ko values. By contrast, when the self-interaction substitutions were incorporated into the IgG4_PE Fc region, high positive ko were achieved with multiple distinct binding regions, indicating low self-interaction among the tested proteins. Additionally, as the R409K substitution was incorporated into the IgG4_PE Fc regions, decreased self-interaction was seen among all tested antibodies.

Additionally, polypeptides comprising the BR-D binding region were analyzed when appended to an IgG4 region with S228P/F234A/L235A substitutions (IgG4_P_FALA), an IgG4_P_FALA with Q274K/Q355R/E419Q Triple mutations (IgG4_P_FALA_Triple), or the IgG4_P_FALA Triple mutations with a R409K mutation (IgG4_P_FALA_Triple_R409K). IgG4_P_FALA exhibited high self-interaction (as seen by low positive results, 0.7). This self-interaction was decreased with the introduction of Q274K/Q355R/E419Q Triple mutations, as well as with Q274K/Q355R/E419Q Triple mutations and R409K.

Polypeptides comprising the BR-B binding region were analyzed when appended to an IgG4 region with an S228P mutation (IgG4_P), IgG4_P with additional Q274K/Q355R/E419Q Triple mutations (IgG4_P_Triple), IgG4_P_Triple with an additional R409K mutation (IgG4_P_Triple_R409K), IgG4_P with additional T256D/T307Q mutations (IgG4_P_DQ), IgG4_P_DQ with additional Q274K/Q355R/E419Q Triple mutations (IgG4_P_DQ_Triple), IgG4_P_DQ_Triple with additional R409K mutation (lgG4_P_DQ_Triple_R409K), IgG4_P with additional M428L/N434S mutations (IgG4PLS), IgG4P_LS with additional Q274K/Q355R/E419Q Triple mutations (IgG4PLSTriple), and gG4_P_LSTriple with additional R409K mutation (IgG4PLSTriple_R409K). gG4_P exhibited high levels of self interaction (−29.4), while introduction of the Triple mutations alone or the Triple mutations together with R409K resulted in a reduced self-interaction (22.4 and 18.2 kD values, respectively). IgG4_P_DQ and IgG4_P_LS both exhibited high levels of self-interaction (−19.3, −10.0, respectively), while introduction of both the Triple mutations and R409K reduced this self interaction (22.5 and 28.6, respectively). The IgG4_P_LS_Triple was also analyzed, and exhibited decreased self-interaction compared to the ggG44P_LS alone (15.8 vs. −10.0, respectively).

These results indicate that the introduction of the Triple mutations alone, or the triple mutations with the R409K mutation, can decrease self-interaction among polypeptides.

TABLE 5
Dynamic Light Scattering Analysis of
IgG4 appended Binding Regions (BR)

| | BR-A | BR-B | BR-C | BR-D | BR-E |
|---|---|---|---|---|---|
| IgG1 | 52.9 | 43.1 | 36.3 | 30.4 | 41.7 |
| IgG4_PE | −6.2 | 1.5 | 6.8 | −5.0 | 2.5 |
| IgG4_PE_Triple | 32 | 29.2 | 30.6 | 27.5 | 47.1 |
| IgG4_PE_Triple_R409K | 31.8 | 19.0 | 34.1 | 24.3 | 41.4 |
| IgG4_PE_Quad | 41.5 | 28.1 | 33.1 | 27.6 | 48.1 |
| IgG4_PE_Quad_R409K | 41.8 | 27.5 | 43.4 | 31.4 | 38.6 |
| IgG4_P_FALA | n.t. | n.t. | n.t. | 0.7 | n.t. |
| IgG4_P_FALA_Triple | n.t. | n.t. | n.t. | 27.8 | n.t. |
| IgG4_P_FALA_Triple_R409K | n.t. | n.t. | n.t. | 21.5 | n.t. |
| IgG4_P | n.t. | −29.4 | n.t. | n.t. | n.t. |
| IgG4_P_Triple | n.t. | 22.4 | n.t. | n.t. | n.t. |
| IgG4_P_Triple_R409K | n.t. | 18.2 | n.t. | n.t. | n.t. |
| IgG4_P_DQ | n.t. | −19.3 | n.t. | n.t. | n.t. |
| IgG4_P_DQ_Triple_R409K | n.t. | 22.5 | n.t. | n.t. | n.t. |
| IgG4_P_LS | n.t. | −10.0 | n.t. | n.t. | n.t. |
| IgG4_P_LS_Triple | n.t. | 15.8 | n.t. | n.t. | n.t. |
| IgG4_P_LS_Triple_R409K | n.t. | 28.6 | n.t. | n.t. | n.t. |

### Example 5: Analysis of Effect of Self-Interaction Substitutions on Protein Aggregation

The manufacture of antibodies requires a number of purification steps that alter the pH of the solution from which the antibody is to be purified. The use of protein A columns, as well as viral inactivation, are two such steps that frequently implement such pH changes. Fc regions of immunoglobulins have high affinity for protein A, allowing for affinity chromatography with protein A columns to purify Fc containing proteins. This affinity is decreased at lower pH, allowing for elution of the Fc containing proteins from the column. Additionally, viral inactivation often occurs at a low pH. These two purification steps are often critical to manufacturing of clinical antibodies. However, IgG4-based proteins are known to frequently have increased aggregation at low pH. This aggregation is potentiated by substitution of the arginine at position 409 in the IgG4 Fc region to lysine (R409K). As the R409K substitution is known to potentiate aggregation of IgG4 based antibodies at low pH, the effect of the self-interaction substitutions on aggregation was analyzed.

The binding region (BR) of BR-A appended to an IgG Fc region, used elsewhere in these examples, exhibited minimal aggregation at low pH. BR-B, BR-C, BR-D, and BR-E, in IgG1 or IgG4 with and without substitutions, were therefore analyzed for their percent aggregation in a protein A elution assay, and BR-D, BR-E, BR-B, and BR-C appended to polypeptides with various Fc regions were assessed in a mock viral inactivation assay.

Following protein A affinity chromatography and low pH elution from the column, percent aggregation of the various samples was analyzed using analytical SEC-HPLC with a GE Superdex200 Increase 5/150 L GL column and PBS pH 7.4 as the mobile phase. Results are shown in Table 6.

Polypeptides comprising the BR-C binding region, BR-D binding region, and BR-E binding region had low percent aggregation with an IgG1 Fc region, which increased when appended to an IgG4 Fc region without an R409K substitution. Introduction of an R409K substitution into the IgG4_PE_Triple and IgG4_PE_Quad Fc regions reduced this aggregation. Polypeptides comprising the BR-D binding region appended to IgG4 regions with S228P/F234A/L235A mutations (IgG4_P_FALA). IgG4_P_FALA mutations with the Q274K/Q355R/E419Q Triple mutations (IgG4_P_FALA_Triple), and the IgG4_P_FALA Triple mutations with a R409K mutation (IgG4_P_FALA Triple_R409K) were also tested. Aggregation levels were similar across treatment groups, though were increased in both the IgG4_P_FALA Triple and IgG4_P_FALA Triple_R409K samples. Polypeptides comprising the BR-B domain appended to IgG4 regions with S228P mutation (IgG4_P), IgG4_P with Q274K/Q355R/E419Q mutations (IgG4_P_Triple), IgG4_P_Triple with an R409K mutation (IgG4_P_Triple_R409K), IgG4_P with M428L/N434S mutations (IgG4_P_LS), IgG4_P_LS with Q274K/Q355R/E419Q mutations (IgG4_P_LS_Triple), and IgG4_P_LS_Triple with an additional R409K mutation (IgG4_P_LS_Triple_R409K) were also tested. Introduction of the Triple Q274K/Q355R/E419Q increased aggregation compared to the IgG4_P alone. Aggregation was reduced when the R409K mutation was introduced into IgG4_P_Triple. The IgG4_P_LS and IgG4_P_LS_Triple showed the same level of aggregation. However, consistent with the presence of the R409K in other IgG4 regions, the R409K substitution reduced aggregation compared to both IgG4_P and IgG4_P_LS.

TABLE 6
Percent Aggregate in Protein A Elution Assay

| | BR-B | BR-C | BR-D | BR-E |
|---|---|---|---|---|
| IgG1 | 15 | 1 | 1 | 1 |
| IgG4_PE | 12 | 14 | 16 | 20 |
| IgG4_PE_Triple | 40 | 37 | 37 | 21 |
| IgG4_PE_Triple_R409K | 16 | 6 | 8 | 6 |
| IgG4_PE_Quad | 50 | 36 | 20 | 51 |
| IgG4_PE_Quad_R409K | 10 | 8 | 9 | 6 |
| IgG4_P_FALA | n.t. | n.t. | 10 | n.t. |
| IgG4_P_FALA_Triple | n.t. | n.t. | 13 | n.t. |
| IgG4_P_FALA_Triple_R409K | n.t. | n.t. | 14 | n.t. |
| IgG4_P | 11 | n.t. | n.t. | n.t. |
| IgG4_P_Triple | 22 | n.t. | n.t. | n.t. |
| IgG4_P_Triple_R409K | 16 | n.t. | n.t. | n.t. |
| IgG4_P_LS | 16 | n.t. | n.t. | n.t. |
| IgG4_P_LS_Triple | 16 | n.t. | n.t. | n.t. |
| IgG4_P_LS_Triple_R409K | 7 | n.t. | n.t. | n.t. |

*n.t. = not tested

A mock viral inactivation assay was used to analyze aggregates. Mock viral inactivation was performed by spiking in 1 M acetic acid into stock solution of antibodies at 1 mg/mL in 10 mM histidine pH 6.0 to get to pH of 3.8 and 3.5. The antibodies were held at low pH for 4 hours and then neutralized to neutral pH using 1M tris base pH 10 buffer. The aggregation of the resulting samples was measured using analytical size exclusion chromatography on a Waters Acquity Ultra-Performance Liquid Chromatography system (UPLC) with a Superdex 200 Increase 5/150 GL column (Cytiva) at 0.3 ml/min with PBS pH 7.2 mobile phase. The experiment was performed in duplicate at each low pH. Results are shown in FIG. 2.

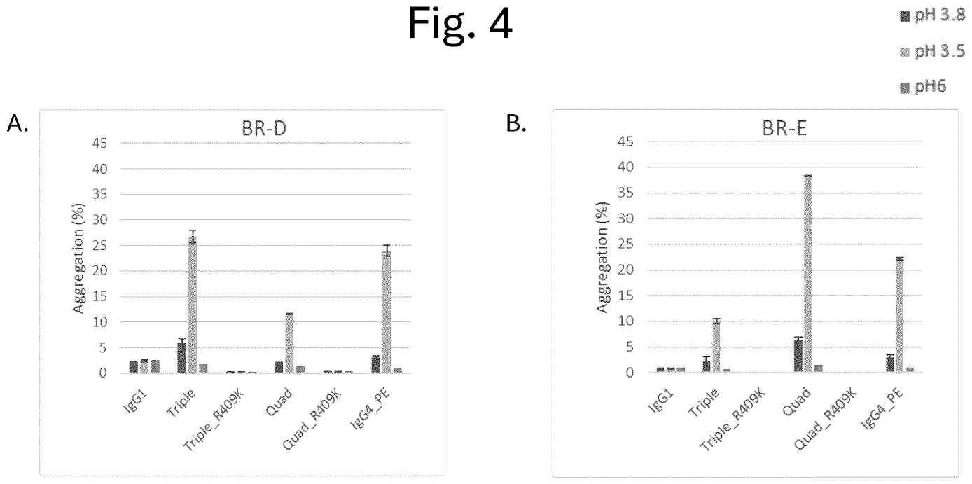

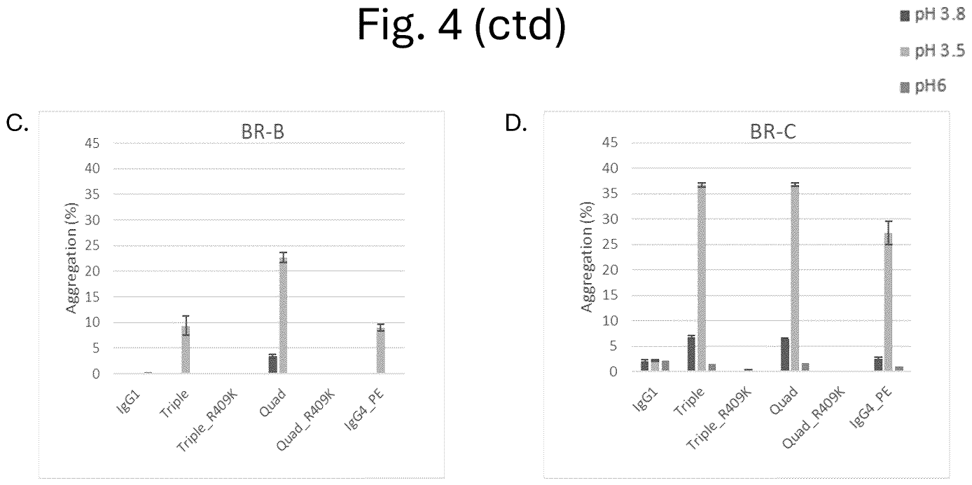

Neither BR-D (FIG. 4A) nor BR-E (FIG. 4B) exhibited significant aggregation at any tested pH when appended to an IgG1 Fc region. Aggregation comparatively increased when both binding regions were appended to an IgG4_PE Fc region. BR-D appended to the IgG4_PE_Triple Fc region exhibited increased aggregation compared to the same binding region appended to the IgG4_PE Fc region. BR-D appended to the IgG4_PE_Quad Fc region exhibited decreased aggregation compared to the same binding region appended to the IgG4_PE Fc region. When the R409K substitution was introduced, aggregation was decreased regardless of Fc region. BR-E appended to the IgG4_PE_Triple Fc region exhibited decreased aggregation compared to the same binding region appended to the IgG4_PE Fc region. BR-E appended to the IgG4_PE_Quad Fc region exhibited increased aggregation compared to the same binding region appended to the IgG4_PE Fc region. As with BR-D samples, BR-E samples exhibited decreased aggregation when the R409K substitution was introduced, regardless of Fc region.

Neither BR-B polypeptides (FIG. 4C) nor BR-C polypeptides (FIG. 4D) exhibited significant aggregation at any tested pH when appended to an IgG1 Fc region. Aggregation comparatively increased when both binding regions were appended to an IgG4_PE Fc region. BR-B binding region appended to the IgG4_PE_Triple Fc region exhibited similar aggregation to the same binding region appended to the IgG4_PE Fc region. BR-B binding region appended to the IgG4_PE_Quad Fc region exhibited increased aggregation compared to the same binding region appended to the IgG4_PE Fc region. When the R409K substitution was introduced, aggregation was decreased regardless of Fc region. BR-C binding region appended to the IgG4_PE_Triple Fc region exhibited increased aggregation compared to the same binding region appended to the IgG4_PE Fc region. BR-C binding region appended to the IgG4_PE_Quad Fc region exhibited increased aggregation compared to the same binding region appended to the IgG4_PE Fc region. As with BR-B samples, BR-C samples exhibited decreased aggregation when the R409K substitution was introduced, regardless of Fc region. These results indicate that the self-interaction substitutions are compatible with the R409K aggregation substitution.

### Example 6. Analysis of Effect of Self-Interaction Substitutions on Protein Yield

Total yield during polypeptide production was analyzed for polypeptides comprising the BR-D binding region appended to IgG4 regions with S228P/F234A/L235A mutations (IgG4_P_FALA). IgG4_P_FALA mutations with the Q274K/Q355R/E419Q Triple mutations (IgG4_P_FALA_Triple), and the IgG4_P_FALA Triple mutations with a R409K mutation (lgG4_P_FALA_Triple_R409K). Polypeptides comprising the BR-B domain appended to IgG4 regions with S228P mutation (IgG4_P), IgG4_P with Q274K/Q355R/E419Q mutations (IgG4_P_Triple), IgG4_P_Triple with an R409K mutation (IgG4_P_Triple_R409K), IgG4_P with M428L/N434S mutations (IgG4_P_LS), IgG4_P_LS with Q274K/Q355R/E419Q mutations (IgG4_P_LS_Triple), and IgG4_P_LS_Triple with an additional R409K mutation (IgG4_P_LS_Triple_R409K) were also analyzed. Results are seen in Table 7. All polypeptides comprising a BR-D domain exhibited high yield, with increased yield observed when appended to the Triple mutations (287 mg/L) or the Triple mutations with an additional R409K mutation (290 mg/L) when compared to the IgG4_P_FALA (270 mg/L). All polypeptides comprising a BR-B binding region exhibited a yield >80 mg/L, except for the IgG_4_P_Triple BR-B polypeptide (55 mg/L).

TABLE 7
Analysis of Protein Yield

| Yield (mg/L) | BR-B | BR-D |
|---|---|---|
| IgG4_P_FALA | n.q. | 270 |
| IgG4_P_FALA_Triple | n.q. | 287 |
| IgG4_P_FALA_Triple_R409K | n.q. | 290 |
| IgG4_P | 80 | n.q. |
| IgG4_P_Triple | 55 | n.q. |
| IgG4_P_Triple_R409K | 80 | n.q. |
| IgG4_P_LS | 82 | n.q. |
| IgG4_P_LS_Triple | 86 | n.q. |
| IgG4_P_LS_Triple_R409K | 80 | n.q. |

*n.q. = not quantified

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All patents and publications cited herein are incorporated by reference herein in their entirety.

### REFERENCES

Zhou et al. Engineered Fc-glycosylation switch to eliminate antibody effector function. MAbs. 2020 January-December; 12(1):1814583.
Kabat et al. Sequences of Proteins of Immunological Interest, National Institutes of Health, Bethesda, MD, 1987 and 1991
Tomar et al. Molecular basis of high viscosity in concentrated antibody solutions: Strategies for high concentration drug product development. MAbs. 2016; 8(2):216-28.
Lai et al. Differences in human IgG1 and IgG4 S228P monoclonal antibodies viscosity and self-interactions: Experimental assessment and computational predictions of domain interactions. MAbs. 2021 January-December; 13(1):1991256.
Kingsbury et al. A single molecular descriptor to predict solution behavior of therapeutic antibodies. Sci Adv. 2020 Aug. 5; 6(32):eabb0372.
Namisaki et al. R409K mutation prevents acid-induced aggregation of human IgG4. PLoS One. 2020 Mar. 17; 15(3):e0229027.
Raut and Kalonia. Opalescence in monoclonal antibody solutions and its correlation with intermolecular interactions in dilute and concentrated solutions. J Pharm Sci. 2015 April; 104(4):1263-74.
Rispens and Huijbers. The unique properties of IgG4 and its roles in health and disease. Nat Rev Immunol 23, 763-778 (2023).
Harmsen and De Haard H J. Properties, production, and applications of camelid single-domain antibody fragments. Appi. Microbiol. Biotechnol. 2007 November; 77(1):13-22.
Schaefer, et al., Immunoglobulin domain crossover as a generic approach for the production of bispecific IgG antibodies. PNAS (2011), 108: 11187-1191
Ridgway, A. A. G. “Mammalian Expression Vectors” Chapter 24.2, pp. 470-472 Vectors, Rodriguez and Denhardt, Eds. (Butterworths, Boston, MA 1988)

TABLE 8
Sequence Listing

| Description/ | | |
|---|---|---|
| SEQ ID NO. | Sequence | |
| Human IgG1 Full | ASTKGPSVFP LAPSSKSTSG GTAALGCLVK DYFPEPVTVS WNSGALTSGV | 50 |
| Constant Region | HTFPAVLQSS GLYSLSSVVT VPSSSLGTQT YICNVNHKPS NTKVDKKVEP | 100 |
| SEQ ID NO: 1 | KSCDKTHTCP PCPAPELLGG PSVFLFPPKP KDTLMISRTP EVTCVVVDVS | 150 |
| | HEDPEVKFNW YVDGVEVHNA KTKPREEQYN STYRVVSVLT VLHQDWLNGK | 200 |
| | EYKCKVSNKA LPAPIEKTIS KAKGQPREPQ VYTLPPSRDE LTKNQVSLTC | 250 |
| | LVKGFYPSDI AVEWESNGQP ENNYKTTPPV LDSDGSFFLY SKLTVDKSRW | 300 |
| | QQGNVFSCSV MHEALHNHYT QKSLSLSPG | 329 |
| IgG1 Fc region | EPKSCDKTHT CPPCPAPELL GGPSVFLFPP KPKDTLMISR TPEVTCVVVD | 50 |
| SEQ ID NO: 2 | VSHEDPEVKF NWYVDGVEVH NAKTKPREEQ YNSTYRVVSV LTVLHQDWLN | 100 |
| | GKEYKCKVSN KALPAPIEKT ISKAKGQPRE PQVYTLPPSR DELTKNQVSL | 150 |
| | TCLVKGFYPS DIAVEWESNG QPENNYKTTP PVLDSDGSFF LYSKLTVDKS | 200 |
| | RWQQGNVFSC SVMHEALHNH YTQKSLSLSP G | 231 |
| Human secreted | ASTKGPSVFP LAPCSRSTSE STAALGCLVK DYFPEPVTVS WNSGALTSGV | 50 |
| IgG4 Full | HTFPAVLQSS GLYSLSSVVT VPSSSLGTKT YTCNVDHKPS NTKVDKRVES | 100 |
| Constant | KYGPPCPSCP APEFLGGPSV FLFPPKPKDT LMISRTPEVT CVVVDVSQED | 150 |
| SEQ ID NO: 3- | PEVQFNWYVD GVEVHNAKTK PREEQFNSTY RVVSVLTVLH QDWLNGKEYK | 200 |
| | CKVSNKGLPS SIEKTISKAK GQPREPQVYT LPPSQEEMTK NQVSLTCLVK | 250 |
| | GFYPSDIAVE WESNGQPENN YKTTPPVLDS DGSFFLYSRL TVDKSRWQEG | 300 |
| | NVFSCSVMHE ALHNHYTQKS LSLSLG | 326 |
| IgG4 Fc only | ESKYGPPCPS CPAPEFLGGP SVFLFPPKPK DTLMISRTPE VTCVVVDVSQ | 50 |
| SEQ ID NO: 4 | EDPEVQFNWY VDGVEVHNAK TKPREEQFNS TYRVVSVLTV LHQDWLNGKE | 100 |
| | YKCKVSNKGL PSSIEKTISK AKGQPREPQV YTLPPSQEEM TKNQVSLTCL | 150 |
| | VKGFYPSDIA VEWESNGQPE NNYKTTPPVL DSDGSFFLYS RLTVDKSRWQ | 200 |
| | EGNVFSCSVM HEALHNHYTQ KSLSLSLG | 228 |
| IgG4_Triple- | ASTKGPSVFP LAPCSRSTSE STAALGCLVK DYFPEPVTVS WNSGALTSGV | 50 |
| Full Constant | HTFPAVLQSS GLYSLSSVVT VPSSSLGTKT YTCNVDHKPS NTKVDKRVES | 100 |
| SEQ ID NO: 5 | KYGPPCPSCP APEFLGGPSV FLFPPKPKDT LMISRTPEVT CVVVDVSQED | 150 |
| | PEVKENWYVD GVEVHNAKTK PREEQFNSTY RVVSVLTVLH QDWLNGKEYK | 200 |
| | CKVSNKGLPS SIEKTISKAK GQPREPQVYT LPPSREEMTK NQVSLTCLVK | 250 |
| | GFYPSDIAVE WESNGQPENN YKTTPPVLDS DGSFFLYSRL TVDKSRWQQG | 300 |
| | NVFSCSVMHE ALHNHYTQKS LSLSLG | 326 |
| IgG4_Quad- | ASTKGPSVFP LAPCSRSTSE STAALGCLVK DYFPEPVTVS WNSGALTSGV | 50 |
| Full Constant | HTFPAVLQSS GLYSLSSVVT VPSSSLGTKT YTCNVDHKPS NTKVDKRVES | 100 |
| SEQ ID NO: 6 | KYGPPCPSCP APEFLGGPSV FLFPPKPKDT LMISRTPEVT CVVVDVSQED | 150 |
| | PEVKFNWYVD GVEVHNAKTK PREQQFNSTY RVVSVLTVLH QDWLNGKEYK | 200 |
| | CKVSNKGLPS SIEKTISKAK GQPREPQVYT LPPSREEMTK NQVSLTCLVK | 250 |
| | GFYPSDIAVE WESNGQPENN YKTTPPVLDS DGSFFLYSRL TVDKSRWQQG | 300 |
| | NVFSCSVMHE ALHNHYTQKS LSLSLG | 326 |
| IgG4_PE_Triple- | ASTKGPSVFP LAPCSRSTSE STAALGCLVK DYFPEPVTVS WNSGALTSGV | 50 |
| Full Constant | HTFPAVLQSS GLYSLSSVVT VPSSSLGTKT YTCNVDHKPS NTKVDKRVES | 100 |
| SEQ ID NO: 7 | KYGPPCPPCP APEFEGGPSV FLFPPKPKDT LMISRTPEVT CVVVDVSQED | 150 |
| | PEVKFNWYVD GVEVHNAKTK PREEQFNSTY RVVSVLTVLH QDWLNGKEYK | 200 |
| | CKVSNKGLPS SIEKTISKAK GQPREPQVYT LPPSREEMTK NQVSLTCLVK | 250 |
| | GFYPSDIAVE WESNGQPENN YKTTPPVLDS DGSFFLYSRL TVDKSRWQQG | 300 |
| | NVFSCSVMHE ALHNHYTQKS LSLSLG | 326 |
| IgG4_PE_Quad- | ASTKGPSVFP LAPCSRSTSE STAALGCLVK DYFPEPVTVS WNSGALTSGV | 50 |
| Full Constant | HTFPAVLQSS GLYSLSSVVT VPSSSLGTKT YTCNVDHKPS NTKVDKRVES | 100 |
| SEQ ID NO: 8 | KYGPPCPPCP APEFEGGPSV FLFPPKPKDT LMISRTPEVT CVVVDVSQED | 150 |
| | PEVKFNWYVD GVEVHNAKTK PREQQFNSTY RVVSVLTVLH QDWLNGKEYK | 200 |
| | CKVSNKGLPS SIEKTISKAK GQPREPQVYT LPPSREEMTK NQVSLTCLVK | 250 |
| | GFYPSDIAVE WESNGQPENN YKTTPPVLDS DGSFFLYSRL TVDKSRWQQG | 300 |
| | NVFSCSVMHE ALHNHYTQKS LSLSLG | 326 |
| IgG4_PE-Full | ASTKGPSVFP LAPCSRSTSE STAALGCLVK DYFPEPVTVS WNSGALTSGV | 50 |
| Constant | HTFPAVLQSS GLYSLSSVVT VPSSSLGTKT YTCNVDHKPS NTKVDKRVES | 100 |
| SEQ ID NO: 9 | KYGPPCPPCP APEFEGGPSV FLFPPKPKDT LMISRTPEVT CVVVDVSQED | 150 |
| | PEVQFNWYVD GVEVHNAKTK PREEQFNSTY RVVSVLTVLH QDWLNGKEYK | 200 |
| | CKVSNKGLPS SIEKTISKAK GQPREPQVYT LPPSQEEMTK NQVSLTCLVK | 250 |
| | GFYPSDIAVE WESNGQPENN YKTTPPVLDS DGSFFLYSRL TVDKSRWQEG | 300 |
| | NVFSCSVMHE ALHNHYTQKS LSLSLG | 326 |
| IgG4_R409K- | ASTKGPSVFP LAPCSRSTSE STAALGCLVK DYFPEPVTVS WNSGALTSGV | 50 |
| Full Constant | HTFPAVLQSS GLYSLSSVVT VPSSSLGTKT YTCNVDHKPS NTKVDKRVES | 100 |
| SEQ ID NO: 10 | KYGPPCPSCP APEFLGGPSV FLFPPKPKDT LMISRTPEVT CVVVDVSQED | 150 |
| | PEVQFNWYVD GVEVHNAKTK PREEQFNSTY RVVSVLTVLH QDWLNGKEYK | 200 |
| | CKVSNKGLPS SIEKTISKAK GQPREPQVYT LPPSQEEMTK NQVSLTCLVK | 250 |
| | GFYPSDIAVE WESNGQPENN YKTTPPVLDS DGSFFLYSKL TVDKSRWQEG | 300 |
| | NVFSCSVMHE ALHNHYTQKS LSLSLG | 326 |
| IgG4_PE_R409K- | ASTKGPSVFP LAPCSRSTSE STAALGCLVK DYFPEPVTVS WNSGALTSGV | 50 |
| Full Constant | HTFPAVLQSS GLYSLSSVVT VPSSSLGTKT YTCNVDHKPS NTKVDKRVES | 100 |
| SEQ ID NO: 11 | KYGPPCPPCP APEFEGGPSV FLFPPKPKDT LMISRTPEVT CVVVDVSQED | 150 |
| | PEVQFNWYVD GVEVHNAKTK PREEQFNSTY RVVSVLTVLH QDWLNGKEYK | 200 |
| | CKVSNKGLPS SIEKTISKAK GQPREPQVYT LPPSQEEMTK NQVSLTCLVK | 250 |
| | GFYPSDIAVE WESNGQPENN YKTTPPVLDS DGSFFLYSKL TVDKSRWQEG | 300 |
| | NVFSCSVMHE ALHNHYTQKS LSLSLG | 326 |
| IgG4_PE_R409K | ASTKGPSVFP LAPCSRSTSE STAALGCLVK DYFPEPVTVS WNSGALTSGV | 50 |
| Triple-Full | HTFPAVLQSS GLYSLSSVVT VPSSSLGTKT YTCNVDHKPS NTKVDKRVES | 100 |
| Constant | KYGPPCPPCP APEFEGGPSV FLFPPKPKDT LMISRTPEVT CVVVDVSQED | 150 |
| SEQ ID NO: 12 | PEVKFNWYVD GVEVHNAKTK PREEQFNSTY RVVSVLTVLH QDWLNGKEYK | 200 |
| | CKVSNKGLPS SIEKTISKAK GQPREPQVYT LPPSREEMTK NQVSLTCLVK | 250 |
| | GFYPSDIAVE WESNGQPENN YKTTPPVLDS DGSFFLYSKL TVDKSRWQQG | 300 |
| | NVFSCSVMHE ALHNHYTQKS LSLSLG | 326 |
| IgG4_PE_R409K | ASTKGPSVFP LAPCSRSTSE STAALGCLVK DYFPEPVTVS WNSGALTSGV | 50 |
| Quad-Full | HTFPAVLQSS GLYSLSSVVT VPSSSLGTKT YTCNVDHKPS NTKVDKRVES | 100 |
| Constant | KYGPPCPPCP APEFEGGPSV FLFPPKPKDT LMISRTPEVT CVVVDVSQED | 150 |
| SEQ ID NO: 13 | PEVKFNWYVD GVEVHNAKTK PREQQFNSTY RVVSVLTVLH QDWLNGKEYK | 200 |
| | CKVSNKGLPS SIEKTISKAK GQPREPQVYT LPPSREEMTK NQVSLTCLVK | 250 |
| | GFYPSDIAVE WESNGQPENN YKTTPPVLDS DGSFFLYSKL TVDKSRWQQG | 300 |
| | NVFSCSVMHE ALHNHYTQKS LSLSLG | 326 |
| IgG4_Triple-Fc | ESKYGPPCPS CPAPEFLGGP SVFLFPPKPK DTLMISRTPE VTCVVVDVSQ | 50 |
| only | EDPEVKFNWY VDGVEVHNAK TKPREEQFNS TYRVVSVLTV LHQDWLNGKE | 100 |
| SEQ ID NO: 14 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_Quad-Fc | ESKYGPPCPS CPAPEFLGGP SVFLFPPKPK DTLMISRTPE VTCVVVDVSQ | 50 |
| only | EDPEVKFNWY VDGVEVHNAK TKPREQQFNS TYRVVSVLTV LHQDWLNGKE | 100 |
| SEQ ID NO: 15 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_PE_Triple- | ESKYGPPCPP CPAPEFEGGP SVFLFPPKPK DTLMISRTPE VTCVVVDVSQ | 50 |
| Fc only | EDPEVKFNWY VDGVEVHNAK TKPREEQFNS TYRVVSVLTV LHQDWLNGKE | 100 |
| SEQ ID NO: 16 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_PE_Quad- | ESKYGPPCPP CPAPEFEGGP SVFLFPPKPK DTLMISRTPE VTCVVVDVSQ | 50 |
| Fc only | EDPEVKFNWY VDGVEVHNAK TKPREQQFNS TYRVVSVLTV LHQDWLNGKE | 100 |
| SEQ ID NO: 17 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_PE-Fc | ESKYGPPCPP CPAPEFEGGP SVFLFPPKPK DTLMISRTPE VTCVVVDVSQ | 50 |
| only | EDPEVQFNWY VDGVEVHNAK TKPREEQFNS TYRVVSVLTV LHQDWLNGKE | 100 |
| SEQ ID NO: 18 | YKCKVSNKGL PSSIEKTISK AKGQPREPQV YTLPPSQEEM TKNQVSLTCL | 150 |
| | VKGFYPSDIA VEWESNGQPE NNYKTTPPVL DSDGSFFLYS RLTVDKSRWQ | 200 |
| | EGNVFSCSVM HEALHNHYTQ KSLSLSLG | 228 |
| IgG4_R409K-Fc | ESKYGPPCPS CPAPEFLGGP SVFLFPPKPK DTLMISRTPE VTCVVVDVSQ | 50 |
| SEQ ID NO: 19 | EDPEVQFNWY VDGVEVHNAK TKPREEQFNS TYRVVSVLTV LHQDWLNGKE | 100 |
| | YKCKVSNKGL PSSIEKTISK AKGQPREPQV YTLPPSQEEM TKNQVSLTCL | 150 |
| | VKGFYPSDIA VEWESNGQPE NNYKTTPPVL DSDGSFFLYS KLTVDKSRWQ | 200 |
| | EGNVFSCSVM HEALHNHYTQ KSLSLSLG | 228 |
| IgG4_PE_R409K- | ESKYGPPCPP CPAPEFEGGP SVFLFPPKPK DTLMISRTPE VTCVVVDVSQ | 50 |
| Fc only | EDPEVQFNWY VDGVEVHNAK TKPREEQFNS TYRVVSVLTV LHQDWLNGKE | 100 |
| SEQ ID NO: 20 | YKCKVSNKGL PSSIEKTISK AKGQPREPQV YTLPPSQEEM TKNQVSLTCL | 150 |
| | VKGFYPSDIA VEWESNGQPE NNYKTTPPVL DSDGSFFLYS KLTVDKSRWQ | 200 |
| | EGNVFSCSVM HEALHNHYTQ KSLSLSLG | 228 |
| IgG4_PE_R409K_ | ESKYGPPCPP CPAPEFEGGP SVFLFPPKPK DTLMISRTPE VTCVVVDVSQ | 50 |
| Triple-Fc only | EDPEVKFNWY VDGVEVHNAK TKPREEQFNS TYRVVSVLTV LHQDWLNGKE | 100 |
| SEQ ID NO: 21 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVM HEALHNHYTQ KSLSLSLG | 228 |
| IgG4_PE_R409K_ | ESKYGPPCPP CPAPEFEGGP SVFLFPPKPK DTLMISRTPE VTCVVVDVSQ | 50 |
| Quad-Fc only | EDPEVKFNWY VDGVEVHNAK TKPREQQFNS TYRVVSVLTV LHQDWLNGKE | 100 |
| SEQ ID NO: 22 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVM HEALHNHYTQ KSLSLSLG | 228 |
| IgG4_P-Full | ASTKGPSVFP LAPCSRSTSE STAALGCLVK DYFPEPVTVS WNSGALTSGV | 50 |
| Constant | HTFPAVLQSS GLYSLSSVVT VPSSSLGTKT YTCNVDHKPS NTKVDKRVES | 100 |
| SEQ ID NO: 23 | KYGPPCPPCP APEFLGGPSV FLFPPKPKDT LMISRTPEVT CVVVDVSQED | 150 |
| | PEVQFNWYVD GVEVHNAKTK PREEQFNSTY RVVSVLTVLH QDWLNGKEYK | 200 |
| | CKVSNKGLPS SIEKTISKAK GQPREPQVYT LPPSQEEMTK NQVSLTCLVK | 250 |
| | GFYPSDIAVE WESNGQPENN YKTTPPVLDS DGSFFLYSRL TVDKSRWQEG | 300 |
| | NVFSCSVMHE ALHNHYTQKS LSLSLG | 326 |
| IgG4_P-Fc only | ESKYGPPCPP CPAPEFLGGP SVFLFPPKPK DTLMISRTPE VTCVVVDVSQ | 50 |
| SEQ ID NO: 24 | EDPEVQFNWY VDGVEVHNAK TKPREEQFNS TYRVVSVLTV LHQDWLNGKE | 100 |
| | YKCKVSNKGL PSSIEKTISK AKGQPREPQV YTLPPSQEEM TKNQVSLTCL | 150 |
| | VKGFYPSDIA VEWESNGQPE NNYKTTPPVL DSDGSFFLYS RLTVDKSRWQ | 200 |
| | EGNVFSCSVM HEALHNHYTQ KSLSLSLG | 228 |
| IgG4_P_Triple- | ASTKGPSVFP LAPCSRSTSE STAALGCLVK DYFPEPVTVS WNSGALTSGV | 50 |
| Full Constant | HTFPAVLQSS GLYSLSSVVT VPSSSLGTKT YTCNVDHKPS NTKVDKRVES | 100 |
| SEQ ID NO: 25 | KYGPPCPPCP APEFLGGPSV FLFPPKPKDT LMISRTPEVT CVVVDVSQED | 150 |
| | PEVKFNWYVD GVEVHNAKTK PREEQFNSTY RVVSVLTVLH QDWLNGKEYK | 200 |
| | CKVSNKGLPS SIEKTISKAK GQPREPQVYT LPPSREEMTK NQVSLTCLVK | 250 |
| | GFYPSDIAVE WESNGQPENN YKTTPPVLDS DGSFFLYSRL TVDKSRWQQG | 300 |
| | NVFSCSVMHE ALHNHYTQKS LSLSLG | 326 |
| IgG4_P_Triple- | ESKYGPPCPP CPAPEFLGGP SVFLFPPKPK DTLMISRTPE VTCVVVDVSQ | 50 |
| Fc only | EDPEVKFNWY VDGVEVHNAK TKPREEQFNS TYRVVSVLTV LHQDWLNGKE | 100 |
| SEQ ID NO: 26 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_P_Quad- | ASTKGPSVFP LAPCSRSTSE STAALGCLVK DYFPEPVTVS WNSGALTSGV | 50 |
| Full Constant | HTFPAVLQSS GLYSLSSVVT VPSSSLGTKT YTCNVDHKPS NTKVDKRVES | 100 |
| SEQ ID NO: 27 | KYGPPCPPCP APEFLGGPSV FLFPPKPKDT LMISRTPEVT CVVVDVSQED | 150 |
| | PEVKFNWYVD GVEVHNAKTK PREQQFNSTY RVVSVLTVLH QDWLNGKEYK | 200 |
| | CKVSNKGLPS SIEKTISKAK GQPREPQVYT LPPSREEMTK NQVSLTCLVK | 250 |
| | GFYPSDIAVE WESNGQPENN YKTTPPVLDS DGSFFLYSRL TVDKSRWQQG | 300 |
| | NVFSCSVMHE ALHNHYTQKS LSLSLG | 326 |
| IgG4_P_Quad- | ESKYGPPCPP CPAPEFLGGP SVFLFPPKPK DTLMISRTPE VTCVVVDVSQ | 50 |
| Fc only | EDPEVKFNWY VDGVEVHNAK TKPREQQFNS TYRVVSVLTV LHQDWLNGKE | 100 |
| SEQ ID NO: 28 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_P_R409K- | ASTKGPSVFP LAPCSRSTSE STAALGCLVK DYFPEPVTVS WNSGALTSGV | 50 |
| Full Constant | HTFPAVLQSS GLYSLSSVVT VPSSSLGTKT YTCNVDHKPS NTKVDKRVES | 100 |
| SEQ ID NO: 29 | KYGPPCPPCP APEFLGGPSV FLFPPKPKDT LMISRTPEVT CVVVDVSQED | 150 |
| | PEVQFNWYVD GVEVHNAKTK PREEQFNSTY RVVSVLTVLH QDWLNGKEYK | 200 |
| | CKVSNKGLPS SIEKTISKAK GQPREPQVYT LPPSQEEMTK NQVSLTCLVK | 250 |
| | GFYPSDIAVE WESNGQPENN YKTTPPVLDS DGSFFLYSKL TVDKSRWQEG | 300 |
| | NVFSCSVMHE ALHNHYTQKS LSLSLG | 326 |
| IgG4_P_R409K- | ESKYGPPCPP CPAPEFLGGP SVFLFPPKPK DTLMISRTPE VTCVVVDVSQ | 50 |
| Fc only | EDPEVQFNWY VDGVEVHNAK TKPREEQFNS TYRVVSVLTV LHQDWLNGKE | 100 |
| SEQ ID NO: 30 | YKCKVSNKGL PSSIEKTISK AKGQPREPQV YTLPPSQEEM TKNQVSLTCL | 150 |
| | VKGFYPSDIA VEWESNGQPE NNYKTTPPVL DSDGSFFLYS KLTVDKSRWQ | 200 |
| | EGNVFSCSVM HEALHNHYTQ KSLSLSLG | 228 |
| IgG4_P_Triple_ | ASTKGPSVFP LAPCSRSTSE STAALGCLVK DYFPEPVTVS WNSGALTSGV | 50 |
| R409K-Full | HTFPAVLQSS GLYSLSSVVT VPSSSLGTKT YTCNVDHKPS NTKVDKRVES | 100 |
| Constant | KYGPPCPPCP APEFLGGPSV FLFPPKPKDT LMISRTPEVT CVVVDVSQED | 150 |
| SEQ ID NO: 31 | PEVKFNWYVD GVEVHNAKTK PREEQFNSTY RVVSVLTVLH QDWLNGKEYK | 200 |
| | CKVSNKGLPS SIEKTISKAK GQPREPQVYT LPPSREEMTK NQVSLTCLVK | 250 |
| | GFYPSDIAVE WESNGQPENN YKTTPPVLDS DGSFFLYSKL TVDKSRWQQG | 300 |
| | NVFSCSVMHE ALHNHYTQKS LSLSLG | 326 |
| IgG4_P_Triple_ | ESKYGPPCPP CPAPEFLGGP SVFLFPPKPK DTLMISRTPE VTCVVVDVSQ | 50 |
| R409K-Fc only | EDPEVKFNWY VDGVEVHNAK TKPREEQFNS TYRVVSVLTV LHQDWLNGKE | 100 |
| SEQ ID NO: 32 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVM HEALHNHYTQ KSLSLSLG | 228 |
| IgG4_P_Quad_ | ASTKGPSVFP LAPCSRSTSE STAALGCLVK DYFPEPVTVS WNSGALTSGV | 50 |
| R409K-Full | HTFPAVLQSS GLYSLSSVVT VPSSSLGTKT YTCNVDHKPS NTKVDKRVES | 100 |
| Constant | KYGPPCPPCP APEFLGGPSV FLFPPKPKDT LMISRTPEVT CVVVDVSQED | 150 |
| SEQ ID NO: 33 | PEVKFNWYVD GVEVHNAKTK PREQQFNSTY RVVSVLTVLH QDWLNGKEYK | 200 |
| | CKVSNKGLPS SIEKTISKAK GQPREPQVYT LPPSREEMTK NQVSLTCLVK | 250 |
| | GFYPSDIAVE WESNGQPENN YKTTPPVLDS DGSFFLYSKL TVDKSRWQQG | 300 |
| | NVFSCSVMHE ALHNHYTQKS LSLSLG | 326 |
| IgG4_P_Quad_ | ESKYGPPCPP CPAPEFLGGP SVFLFPPKPK DTLMISRTPE VTCVVVDVSQ | 50 |
| R409K-Fc only | EDPEVKFNWY VDGVEVHNAK TKPREQQFNS TYRVVSVLTV LHQDWLNGKE | 100 |
| SEQ ID NO: 34 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVM HEALHNHYTQ KSLSLSLG | 228 |
| IgG4_P_FALA | ESKYGPPCPP CPAPEAAGGP SVFLFPPKPK DTLMISRTPE VTCVVVDVSQ | 50 |
| SEQ ID NO: 35 | EDPEVQFNWY VDGVEVHNAK TKPREEQFNS TYRVVSVLTV LHQDWLNGKE | 100 |
| | YKCKVSNKGL PSSIEKTISK AKGQPREPQV YTLPPSQEEM TKNQVSLTCL | 150 |
| | VKGFYPSDIA VEWESNGQPE NNYKTTPPVL DSDGSFFLYS RLTVDKSRWQ | 200 |
| | EGNVFSCSVM HEALHNHYTQ KSLSLSLG | 228 |
| IgG4_P_FALA_ | ESKYGPPCPP CPAPEAAGGP SVFLFPPKPK DTLMISRTPE VTCVVVDVSQ | 50 |
| Triple | EDPEVKFNWY VDGVEVHNAK TKPREEQFNS TYRVVSVLTV LHQDWLNGKE | 100 |
| SEQ ID NO: 36 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_P_FALA_ | ESKYGPPCPP CPAPEAAGGP SVFLFPPKPK DTLMISRTPE VTCVVVDVSQ | 50 |
| Triple_R409K | EDPEVKFNWY VDGVEVHNAK TKPREEQFNS TYRVVSVLTV LHQDWLNGKE | 100 |
| SEQ ID NO: 37 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVM HEALHNHYTQ KSLSLSLG | 228 |
| IgG4_P_FALA_ | ESKYGPPCPP CPAPEAAGGP SVFLFPPKPK DTLMISRTPE VTCVVVDVSQ | 50 |
| Quad | EDPEVKFNWY VDGVEVHNAK TKPREQQFNS TYRVVSVLTV LHQDWLNGKE | 100 |
| SEQ ID NO: 38 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_P_FALA_ | ESKYGPPCPP CPAPEAAGGP SVFLFPPKPK DTLMISRTPE VTCVVVDVSQ | 50 |
| Quad_R409K | EDPEVKFNWY VDGVEVHNAK TKPREQQFNS TYRVVSVLTV LHQDWLNGKE | 100 |
| SEQ ID NO: 39 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVM HEALHNHYTQ KSLSLSLG | 228 |
| IgG4_P_DQ | ESKYGPPCPP CPAPEFLGGP SVFLFPPKPK DTLMISRDPE VTCVVVDVSQ | 50 |
| SEQ ID NO: 40 | EDPEVQFNWY VDGVEVHNAK TKPREEQFNS TYRVVSVLQV LHQDWLNGKE | 100 |
| | YKCKVSNKGL PSSIEKTISK AKGQPREPQV YTLPPSQEEM TKNQVSLTCL | 150 |
| | VKGFYPSDIA VEWESNGQPE NNYKTTPPVL DSDGSFFLYS RLTVDKSRWQ | 200 |
| | EGNVFSCSVM HEALHNHYTQ KSLSLSLG | 228 |
| IgG4_P_DQ_ | ESKYGPPCPP CPAPEFLGGP SVFLFPPKPK DTLMISRDPE VTCVVVDVSQ | 50 |
| Triple | EDPEVKFNWY VDGVEVHNAK TKPREEQFNS TYRVVSVLQV LHQDWLNGKE | 100 |
| SEQ ID NO: 41 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_P_DQ_ | ESKYGPPCPP CPAPEFLGGP SVFLFPPKPK DTLMISRDPE VTCVVVDVSQ | 50 |
| Triple_R409K | EDPEVKFNWY VDGVEVHNAK TKPREEQFNS TYRVVSVLQV LHQDWLNGKE | 100 |
| SEQ ID NO: 42 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVM HEALHNHYTQ KSLSLSLG | 228 |
| IgG4_P_DQ_ | ESKYGPPCPP CPAPEFLGGP SVFLFPPKPK DTLMISRDPE VTCVVVDVSQ | 50 |
| Quad | EDPEVKFNWY VDGVEVHNAK TKPREQQFNS TYRVVSVLQV LHQDWLNGKE | 100 |
| SEQ ID NO: 43 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_P_DQ_ | ESKYGPPCPP CPAPEFLGGP SVFLFPPKPK DTLMISRDPE VTCVVVDVSQ | 50 |
| Quad_R409K | EDPEVKFNWY VDGVEVHNAK TKPREQQFNS TYRVVSVLQV LHQDWLNGKE | 100 |
| SEQ ID NO: 44 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVM HEALHNHYTQ KSLSLSLG | 228 |
| IgG4_P_LS | ESKYGPPCPP CPAPEFLGGP SVFLFPPKPK DTLMISRTPE VTCVVVDVSQ | 50 |
| SEQ ID NO: 45 | EDPEVQFNWY VDGVEVHNAK TKPREEQFNS TYRVVSVLTV LHQDWLNGKE | 100 |
| | YKCKVSNKGL PSSIEKTISK AKGQPREPQV YTLPPSQEEM TKNQVSLTCL | 150 |
| | VKGFYPSDIA VEWESNGQPE NNYKTTPPVL DSDGSFFLYS RLTVDKSRWQ | 200 |
| | EGNVFSCSVL HEALHSHYTQ KSLSLSLG | 228 |
| IgG4_P_LS_ | ESKYGPPCPP CPAPEFLGGP SVFLFPPKPK DTLMISRTPE VTCVVVDVSQ | 50 |
| Triple | EDPEVKFNWY VDGVEVHNAK TKPREEQFNS TYRVVSVLTV LHQDWLNGKE | 100 |
| SEQ ID NO: 46 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVL HEALHSHYTQ KSLSLSLG | 228 |
| IgG4_P_LS_ | ESKYGPPCPP CPAPEFLGGP SVFLFPPKPK DTLMISRTPE VTCVVVDVSQ | 50 |
| Triple_R409K | EDPEVKFNWY VDGVEVHNAK TKPREEQFNS TYRVVSVLTV LHQDWLNGKE | 100 |
| SEQ ID NO: 47 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVL HEALHSHYTQ KSLSLSLG | 228 |
| IgG4_P_LS_ | ESKYGPPCPP CPAPEFLGGP SVFLFPPKPK DTLMISRTPE VTCVVVDVSQ | 50 |
| Quad | EDPEVKFNWY VDGVEVHNAK TKPREQQFNS TYRVVSVLTV LHQDWLNGKE | 100 |
| SEQ ID NO: 48 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVL HEALHSHYTQ KSLSLSLG | 228 |
| IgG4_P_LS_ | ESKYGPPCPP CPAPEFLGGP SVFLFPPKPK DTLMISRTPE VTCVVVDVSQ | 50 |
| Quad_R409K | EDPEVKFNWY VDGVEVHNAK TKPREQQFNS TYRVVSVLTV LHQDWLNGKE | 100 |
| SEQ ID NO: 49 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVL HEALHSHYTQ KSLSLSLG | 228 |
| IgG4_PE_DQ_ | ESKYGPPCPP CPAPEFEGGP SVFLFPPKPK DTLMISRDPE VTCVVVDVSQ | 50 |
| Triple | EDPEVKFNWY VDGVEVHNAK TKPREEQFNS TYRVVSVLQV LHQDWLNGKE | 100 |
| SEQ ID NO: 50 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_PE_DQ_ | ESKYGPPCPP CPAPEFEGGP SVFLFPPKPK DTLMISRDPE VTCVVVDVSQ | 50 |
| Quad | EDPEVKFNWY VDGVEVHNAK TKPREQQFNS TYRVVSVLQV LHQDWLNGKE | 100 |
| SEQ ID NO: 51 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_PE_DQ_ | ESKYGPPCPP CPAPEFEGGP SVFLFPPKPK DTLMISRDPE VTCVVVDVSQ | 50 |
| Triple_R409K | EDPEVKFNWY VDGVEVHNAK TKPREEQFNS TYRVVSVLQV LHQDWLNGKE | 100 |
| SEQ ID NO: 52 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVM HEALHNHYTQ KSLSLSLG | 228 |
| IgG4_PE_DQ_ | ESKYGPPCPP CPAPEFEGGP SVFLFPPKPK DTLMISRDPE VTCVVVDVSQ | 50 |
| Quad_R409K | EDPEVKFNWY VDGVEVHNAK TKPREQQFNS TYRVVSVLQV LHQDWLNGKE | 100 |
| SEQ ID NO: 53 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVM HEALHNHYTQ KSLSLSLG | 228 |
| IgG4_PE_LS_ | ESKYGPPCPP CPAPEFEGGP SVFLFPPKPK DTLMISRTPE VTCVVVDVSQ | 50 |
| Triple | EDPEVKFNWY VDGVEVHNAK TKPREEQFNS TYRVVSVLTV LHQDWLNGKE | 100 |
| SEQ ID NO: 54 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVL HEALHSHYTQ KSLSLSLG | 228 |
| IgG4_PE_LS_ | ESKYGPPCPP CPAPEFEGGP SVFLFPPKPK DTLMISRTPE VTCVVVDVSQ | 50 |
| Triple_R409K | EDPEVKFNWY VDGVEVHNAK TKPREEQFNS TYRVVSVLTV LHQDWLNGKE | 100 |
| SEQ ID NO: 55 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVL HEALHSHYTQ KSLSLSLG | 228 |
| IgG4_PE_LS_ | ESKYGPPCPP CPAPEFEGGP SVFLFPPKPK DTLMISRTPE VTCVVVDVSQ | 50 |
| Quad | EDPEVKFNWY VDGVEVHNAK TKPREQQFNS TYRVVSVLTV LHQDWLNGKE | 100 |
| SEQ ID NO: 56 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVL HEALHSHYTQ KSLSLSLG | 228 |
| IgG4_PE_LS_ | ESKYGPPCPP CPAPEFEGGP SVFLFPPKPK DTLMISRTPE VTCVVVDVSQ | 50 |
| Quad_R409K | EDPEVKFNWY VDGVEVHNAK TKPREQQFNS TYRVVSVLTV LHQDWLNGKE | 100 |
| SEQ ID NO: 57 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVL HEALHSHYTQ KSLSLSLG | 228 |
| IgG4_P_DQ_ | ESKYGPPCPP CPAPEAAGGP SVFLFPPKPK DTLMISRDPE VTCVVVDVSQ | 50 |
| FALA_Triple | EDPEVKFNWY VDGVEVHNAK TKPREEQFNS TYRVVSVLQV LHQDWLNGKE | 100 |
| SEQ ID NO: 58 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_P_DQ_ | ESKYGPPCPP CPAPEAAGGP SVFLFPPKPK DTLMISRDPE VTCVVVDVSQ | 50 |
| FALA_Triple_ | EDPEVKFNWY VDGVEVHNAK TKPREEQFNS TYRVVSVLQV LHQDWLNGKE | 100 |
| R409K | YKCKVSNKGL PSSIEKTISK AKGQPREPQV YTLPPSREEM TKNQVSLTCL | 150 |
| SEQ ID NO: 59 | VKGFYPSDIA VEWESNGQPE NNYKTTPPVL DSDGSFFLYS KLTVDKSRWQ | 200 |
| | QGNVFSCSVM HEALHNHYTQ KSLSLSLG | 228 |
| IgG4_P_DQ_ | ESKYGPPCPP CPAPEAAGGP SVFLFPPKPK DTLMISRDPE VTCVVVDVSQ | 50 |
| FALA_Quad | EDPEVKFNWY VDGVEVHNAK TKPREQQFNS TYRVVSVLQV LHQDWLNGKE | 100 |
| SEQ ID NO: 60 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVM HEALHNHYTQ KSLSLSLG | 228 |
| IgG4_P_DQ_ | ESKYGPPCPP CPAPEAAGGP SVFLFPPKPK DTLMISRDPE VTCVVVDVSQ | 50 |
| FALA_Quad_ | EDPEVKFNWY VDGVEVHNAK TKPREQQFNS TYRVVSVLQV LHQDWLNGKE | 100 |
| R409K | YKCKVSNKGL PSSIEKTISK AKGQPREPQV YTLPPSREEM TKNQVSLTCL | 150 |
| SEQ ID NO: 61 | VKGFYPSDIA VEWESNGQPE NNYKTTPPVL DSDGSFFLYS KLTVDKSRWQ | 200 |
| | QGNVFSCSVM HEALHNHYTQ KSLSLSLG | 228 |
| IgG4_P_FALA_ | ESKYGPPCPP CPAPEAAGGP SVFLFPPKPK DTLMISRTPE VTCVVVDVSQ | 50 |
| LS | EDPEVQFNWY VDGVEVHNAK TKPREEQFNS TYRVVSVLTV LHQDWLNGKE | 100 |
| SEQ ID NO: 62 | YKCKVSNKGL PSSIEKTISK AKGQPREPQV YTLPPSQEEM TKNQVSLTCL | 150 |
| | VKGFYPSDIA VEWESNGQPE NNYKTTPPVL DSDGSFFLYS RLTVDKSRWQ | 200 |
| | EGNVFSCSVL HEALHSHYTQ KSLSLSLG | 228 |
| IgG4_P_FALA_ | ESKYGPPCPP CPAPEAAGGP SVFLFPPKPK DTLMISRTPE VTCVVVDVSQ | 50 |
| LS_Triple | EDPEVKFNWY VDGVEVHNAK TKPREEQFNS TYRVVSVLTV LHQDWLNGKE | 100 |
| SEQ ID NO: 63 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVL HEALHSHYTQ KSLSLSLG | 228 |
| IgG4_P_FALA_ | ESKYGPPCPP CPAPEAAGGP SVFLFPPKPK DTLMISRTPE VTCVVVDVSQ | 50 |
| LS_Triple_ | EDPEVKFNWY VDGVEVHNAK TKPREEQFNS TYRVVSVLTV LHQDWLNGKE | 100 |
| R409K | YKCKVSNKGL PSSIEKTISK AKGQPREPQV YTLPPSREEM TKNQVSLTCL | 150 |
| SEQ ID NO: 64 | VKGFYPSDIA VEWESNGQPE NNYKTTPPVL DSDGSFFLYS KLTVDKSRWQ | 200 |
| | QGNVFSCSVL HEALHSHYTQ KSLSLSLG | 228 |
| IgG4_P_FALA_ | ESKYGPPCPP CPAPEAAGGP SVFLFPPKPK DTLMISRTPE VTCVVVDVSQ | 50 |
| LS_Quad | EDPEVKFNWY VDGVEVHNAK TKPREQQFNS TYRVVSVLTV LHQDWLNGKE | 100 |
| SEQ ID NO: 65 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS RLTVDKSRWQ | 200 |
| | QGNVFSCSVL HEALHSHYTQ KSLSLSLG | 228 |
| IgG4_P_FALA_ | ESKYGPPCPP CPAPEAAGGP SVFLFPPKPK DTLMISRTPE VTCVVVDVSQ | 50 |
| LS_Quad_R409K | EDPEVKFNWY VDGVEVHNAK TKPREQQFNS TYRVVSVLTV LHQDWLNGKE | 100 |
| SEQ ID NO: 66 | YKCKVSNKGL PSSIEKTISK AKGQPREPQV YTLPPSREEM TKNQVSLTCL | 150 |
| | VKGFYPSDIA VEWESNGQPE NNYKTTPPVL DSDGSFFLYS KLTVDKSRWQ | 200 |
| | QGNVFSCSVL HEALHSHYTQ KSLSLSLG | 228 |

1. An isolated polypeptide comprising an Fc region from human IgG4, wherein the Fc region comprises substitutions at amino acid residues corresponding to amino acid residues Q274, Q355, and E419 according to EU numbering in human IgG4.

2. The isolated polypeptide of claim 1, wherein the glutamine at amino acid residue 274 according to EU numbering in human IgG4 is substituted by lysine (Q274K).

3. The isolated polypeptide of claim 1, wherein the glutamine at amino acid residue 355 according to EU numbering in human IgG4 is substituted with arginine (Q355R).

4. The isolated polypeptide of claim 1, wherein the glutamate at amino acid residue 419 according to EU numbering in human IgG4 is substituted by glutamine (E419Q).

5. The isolated polypeptide of claim 1, wherein the glutamine at amino acid residue 274 according to EU numbering in human IgG4 is substituted by lysine (Q274K); wherein the glutamine at amino acid residue 355 according to EU numbering in human IgG4 is substituted with arginine (Q355R); and wherein the glutamate at amino acid residue 419 according to EU numbering in human IgG4 is substituted by glutamine (E419Q).

6. The isolated polypeptide of claim 5, wherein the Fc region further comprises a substitution at an amino acid residue corresponding to E294 according to EU numbering in human IgG4.

7. The isolated polypeptide of claim 6, wherein the glutamate at amino acid residue 294 according to EU numbering in human IgG4 is substituted by glutamine (E294Q).

8. The isolated polypeptide of claim 1, wherein the glutamine at amino acid residue 274 according to EU numbering in human IgG4 is substituted by lysine (Q274K); wherein the glutamate at amino acid residue 294 according to EU numbering in human IgG4 is substituted by glutamine (E294Q); wherein the glutamine at amino acid residue 355 according to EU numbering in human IgG4 is substituted with arginine (Q355R); and wherein the glutamate at amino acid residue 419 according to EU numbering in human IgG4 is substituted by glutamine (E419Q).

9-23. (canceled)

24. The isolated polypeptide of claim 1, wherein the isolated polypeptide has a diffusion interaction parameter equal to or greater than the diffusion interaction parameter of an Fc region from a wild-type IgG1 polypeptide.

25-35. (canceled)

36. A composition or solution comprising the isolated polypeptide of claim 1, wherein the composition or solution is less viscous than a composition or solution comprising an isolated polypeptide comprising a wildtype Fc region from human IgG4.

37-46. (canceled)

47. A pharmaceutical composition comprising the isolated polypeptide of claim 1 and a pharmaceutically acceptable carrier.

48. A method of treating a disease or disorder, the method comprising administering an effective amount of the isolated polypeptide of claim 1 to a subject in need thereof.

49. A polynucleotide or a set of polynucleotides encoding the isolated polypeptide of claim 1.

50. A vector comprising the polynucleotide or set of polynucleotides of claim 49 and one or more promoter operably linked to the polynucleotide or the set of polynucleotides.

51. A host cell comprising the polynucleotide of claim 49.

52. (canceled)

53. A method of making the isolated polypeptide of claim 1, the method comprising transfecting one or more host cell with a polynucleotide or vector encoding the polypeptide and expressing the polypeptide in the host cell.

54. An isolated polypeptide comprising an amino acid sequence selected from the group consisting of SEQ ID NOs: 5-9, 12-17, 21, 22, 25-28, 31-34, 36-39, 41-44, and 46-66.

55. The isolated polypeptide of claim 5, wherein the serine at amino acid residue 228 according to EU numbering in human IgG4 is substituted by proline (S228P); wherein the threonine at an amino acid residue corresponding to residue 256 in human IgG4 according to EU numbering is substituted with aspartic acid (T256D); and wherein the threonine at an amino acid residue corresponding to residue 307 in human IgG4 according to EU numbering is substituted with glutamine (T307Q).

56-62. (canceled)

63. The isolated polypeptide of claim 5, wherein the serine at amino acid residue 228 according to EU numbering in human IgG4 is substituted by proline (S228P); wherein the methionine at an amino acid residue corresponding to residue 428 in human IgG4 according to EU numbering is substituted with leucine (M428L); and wherein the asparagine at an amino acid residue corresponding to residue 434 in human IgG4 according to EU numbering is substituted with serine (N434S).

64-70. (canceled)

71. An isolated polypeptide comprising an Fc region from human IgG4, wherein the glutamine at amino acid residue 274 according to EU numbering in human IgG4 is substituted by lysine (Q274K); wherein the glutamine at amino acid residue 355 according to EU numbering in human IgG4 is substituted with arginine (Q355R); wherein the glutamate at amino acid residue 419 according to EU numbering in human IgG4 is substituted by glutamine (E419Q); wherein the serine at amino acid residue 228 according to EU numbering in human IgG4 is substituted by proline (S228P); and wherein the phenylalanine at amino acid residue 234 according to EU numbering in human IgG4 is substituted by alanine (F234A) and the leucine at amino acid residue 235 according to EU numbering in human IgG4 is substituted by alanine (L235A).

72-107. (canceled)


SEQUENCE LISTING (66 sequences)

SEQ ID NO: 1 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 4 - protein, 228 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 5 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 6 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 7 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 8 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 9 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 10 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 11 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 12 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 13 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 14 - protein, 228 aa, synthetic construct
ESKYGPPCPSCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 15 - protein, 228 aa, synthetic construct
ESKYGPPCPSCPAPEFLGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 16 - protein, 228 aa, synthetic construct
ESKYGPPCPPCPAPEFEGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 17 - protein, 228 aa, synthetic construct
ESKYGPPCPPCPAPEFEGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 18 - protein, 228 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - protein, 228 aa, synthetic construct
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 20 - protein, 228 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 21 - protein, 228 aa, synthetic construct
ESKYGPPCPPCPAPEFEGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 22 - protein, 228 aa, synthetic construct
ESKYGPPCPPCPAPEFEGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 23 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 24 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 25 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 26 - protein, 228 aa, synthetic construct
ESKYGPPCPPCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 27 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 28 - protein, 228 aa, synthetic construct
ESKYGPPCPPCPAPEFLGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 29 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 30 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 31 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 32 - protein, 228 aa, synthetic construct
ESKYGPPCPPCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 33 - protein, 326 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 34 - protein, 228 aa, synthetic construct
ESKYGPPCPPCPAPEFLGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 35 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 36 - protein, 228 aa, synthetic construct
ESKYGPPCPPCPAPEAAGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 37 - protein, 228 aa, synthetic construct
ESKYGPPCPPCPAPEAAGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 38 - protein, 228 aa, synthetic construct
ESKYGPPCPPCPAPEAAGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 39 - protein, 228 aa, synthetic construct
ESKYGPPCPPCPAPEAAGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 40 - protein, 228 aa, synthetic construct
ESKYGPPCPPCPAPEFLGGPSVFLFPPKPKDTLMISRDPEVTCVVVDVSQEDPEVQFNWYVDGVEVHNAKTKPREEQFNSTYRVVSVLQVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 41 - protein, 228 aa, synthetic construct
ESKYGPPCPPCPAPEFLGGPSVFLFPPKPKDTLMISRDPEVTCVVVDVSQEDPEVKFNWYVDGVEVHNAKTKPREEQFNSTYRVVSVLQVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 42 - protein, 228 aa, synthetic construct
ESKYGPPCPPCPAPEFLGGPSVFLFPPKPKDTLMISRDPEVTCVVVDVSQEDPEVKFNWYVDGVEVHNAKTKPREEQFNSTYRVVSVLQVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 43 - protein, 228 aa, synthetic construct
ESKYGPPCPPCPAPEFLGGPSVFLFPPKPKDTLMISRDPEVTCVVVDVSQEDPEVKFNWYVDGVEVHNAKTKPREQQFNSTYRVVSVLQVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 44 - protein, 228 aa, synthetic construct
ESKYGPPCPPCPAPEFLGGPSVFLFPPKPKDTLMISRDPEVTCVVVDVSQEDPEVKFNWYVDGVEVHNAKTKPREQQFNSTYRVVSVLQVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 45 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 46 - protein, 228 aa, synthetic construct
ESKYGPPCPPCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSLG

SEQ ID NO: 47 - protein, 228 aa, synthetic construct
ESKYGPPCPPCPAPEFLGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSLG

SEQ ID NO: 48 - protein, 228 aa, synthetic construct
ESKYGPPCPPCPAPEFLGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSLG

SEQ ID NO: 49 - protein, 228 aa, synthetic construct
ESKYGPPCPPCPAPEFLGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSLG

SEQ ID NO: 50 - protein, 228 aa, synthetic construct
ESKYGPPCPPCPAPEFEGGPSVFLFPPKPKDTLMISRDPEVTCVVVDVSQEDPEVKFNWYVDGVEVHNAKTKPREEQFNSTYRVVSVLQVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 51 - protein, 228 aa, synthetic construct
ESKYGPPCPPCPAPEFEGGPSVFLFPPKPKDTLMISRDPEVTCVVVDVSQEDPEVKFNWYVDGVEVHNAKTKPREQQFNSTYRVVSVLQVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 52 - protein, 228 aa, synthetic construct
ESKYGPPCPPCPAPEFEGGPSVFLFPPKPKDTLMISRDPEVTCVVVDVSQEDPEVKFNWYVDGVEVHNAKTKPREEQFNSTYRVVSVLQVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 53 - protein, 228 aa, synthetic construct
ESKYGPPCPPCPAPEFEGGPSVFLFPPKPKDTLMISRDPEVTCVVVDVSQEDPEVKFNWYVDGVEVHNAKTKPREQQFNSTYRVVSVLQVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 54 - protein, 228 aa, synthetic construct
ESKYGPPCPPCPAPEFEGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSLG

SEQ ID NO: 55 - protein, 228 aa, synthetic construct
ESKYGPPCPPCPAPEFEGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSLG

SEQ ID NO: 56 - protein, 228 aa, synthetic construct
ESKYGPPCPPCPAPEFEGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSLG

SEQ ID NO: 57 - protein, 228 aa, synthetic construct
ESKYGPPCPPCPAPEFEGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSLG

SEQ ID NO: 58 - protein, 228 aa, synthetic construct
ESKYGPPCPPCPAPEAAGGPSVFLFPPKPKDTLMISRDPEVTCVVVDVSQEDPEVKFNWYVDGVEVHNAKTKPREEQFNSTYRVVSVLQVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 59 - protein, 228 aa, synthetic construct
ESKYGPPCPPCPAPEAAGGPSVFLFPPKPKDTLMISRDPEVTCVVVDVSQEDPEVKFNWYVDGVEVHNAKTKPREEQFNSTYRVVSVLQVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 60 - protein, 228 aa, synthetic construct
ESKYGPPCPPCPAPEAAGGPSVFLFPPKPKDTLMISRDPEVTCVVVDVSQEDPEVKFNWYVDGVEVHNAKTKPREQQFNSTYRVVSVLQVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSLG

SEQ ID NO: 61 - protein, 228 aa, synthetic construct
ESKYGPPCPPCPAPEAAGGPSVFLFPPKPKDTLMISRDPEVTCVVVDVSQEDPEVKFNWYVDGVEVHNAKTKPREQQFNSTYRVVSVLQVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSLG

SEQ ID NO: 62 - protein, 228 aa, synthetic construct
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 63 - protein, 228 aa, synthetic construct
ESKYGPPCPPCPAPEAAGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSLG

SEQ ID NO: 64 - protein, 228 aa, synthetic construct
ESKYGPPCPPCPAPEAAGGPSVFLFPPKPKDTLMISRTPEVTCVVVDVSQEDPEVKFNWYVDGVEVHNAKTKPREEQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSLG

SEQ ID NO: 65 - protein, 228 aa, synthetic construct
ESKYGPPCPPCPAPEAAGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVLHEALHSHYTQKSLSLSLG

SEQ ID NO: 66 - protein, 228 aa, synthetic construct
ESKYGPPCPPCPAPEAAGGPSVFLFPPKPKDTLMISRTPEVTCVVVDVSQEDPEVKFNWYVDGVEVHNAKTKPREQQFNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSLG